SEQUENCE LISTING (8 sequences)

SEQ ID NO: 1 - DNA, 24 bp, Artificial
  primer
tactcttggatcggaaacccgtcg

SEQ ID NO: 2 - DNA, 20 bp, Artificial
  primer
ctactttacttgccaattcc

SEQ ID NO: 3 - DNA, 5693 bp, Artificial
  vector pSVIRES-G
gcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgctctagcccaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatgggcagcgaaaaatacatcgtcacctgggacatgttgcagatccatgcacgtaaactcgcaagccgactgatgccttctgaacaatggaaaggcattattgccgtaagccgtggcggtctggtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 4 - DNA, 544 bp, Artificial
  IRES element
taacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacac

SEQ ID NO: 5 - DNA, 7440 bp, polio virus 1 Mahoney
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatgggtgctcaggtttcatcacagaaagtgggcgcacatgaaaactcaaatagagcgtatggtggttctaccattaattacaccaccattaattattatagagattcagctagtaacgcggcttcgaaacaggacttctctcaagacccttccaagttcaccgagcccatcaaggatgtcctgataaaaacagccccaatgctaaactcgccaaacatagaggcttgcgggtatagcgatagagtactgcaattaacactgggaaactccactataaccacacaggaggcggctaattcagtagtcgcttatgggcgttggcctgaatatctgagggacagcgaagccaatccagtggaccagccgacagaaccagacgtcgctgcatgcaggttttatacgctagacaccgtgtcttggacgaaagagtcgcgagggtggtggtggaagttgcctgatgcactgagggacatgggactctttgggcaaaatatgtactaccactacctaggtaggtccgggtacaccgtgcatgtacagtgtaacgcctccaaattccaccagggggcactaggggtattcgccgtaccagagatgtgtctggccggggatagcaacaccactaccatgcacaccagctatcaaaatgccaatcctggcgagaaaggaggcactttcacgggtacgttcactcctgacaacaaccagacatcacctgcccgcaggttctgcccggtggattacctccttggaaatggcacgttgttggggaatgcctttgtgttcccgcaccagataataaacctacggaccaacaactgtgctacactggtactcccttacgtgaactccctctcgatagatagtatggtaaagcacaataattggggaattgcaatattaccattggccccattaaattttgctagtgagtcctccccagagattccaatcaccttgaccatagcccctatgtgctgtgagttcaatggattaagaaacatcaccctgccacgcttacagggcctgccggtcatgaacacccctggtagcaatcaatatcttactgcagacaacttccagtcaccgtgtgcgctgcctgaatttgatgtgaccccacctattgacatacccggtgaagtaaagaacatgatggaattggcagaaatcgacaccatgattccctttgacttaagtgccacaaaaaagaacaccatggaaatgtatagggttcggttaagtgacaaaccacatacagacgatcccatactctgcctgtcactctctccagcttcagatcctaggttgtcacatactatgcttggagaaatcctaaattactacacacactgggcaggatccctgaagttcacgtttctgttctgtggattcatgatggcaactggcaaactgttggtgtcatacgcgcctcctggagccgacccaccaaagaagcgtaaggaggcgatgttgggaacacatgtgatctgggacataggactgcagtcctcatgtactatggtagtgccatggattagcaacaccacgtatcggcaaaccatagatgatagtttcaccgaaggcggatacatcagcgtcttctaccaaactagaatagtcgtccctctttcgacacccagagagatggacatccttggttttgtgtcagcgtgtaatgacttcagcgtgcgcttgttgcgagataccacacatatagagcaaaaagcgctagcacaggggttaggtcagatgcttgaaagcatgattgacaacacagtccgtgaaacggtgggggcggcaacatctagagacgctctcccaaacactgaagccagtggaccaacacactccaaggaaattccggcactcaccgcagtggaaactggggccacaaatccactagtcccttctgatacagtgcaaaccagacatgttgtacaacataggtcaaggtcagagtctagcatagagtctttcttcgcgcggggtgcatgcgtgaccattatgaccgtggataacccagcttccaccacgaataaggataagctatttgcagtgtggaagatcacttataaagatactgtccagttacggaggaaattggagttcttcacctattctagatttgatatggaacttacctttgtggttactgcaaatttcactgagactaacaatgggcatgccttaaatcaagtgtaccaaattatgtacgtaccaccaggcgctccagtgcccgagaaatgggacgactacacatggcaaacctcatcaaatccatcaatcttttacacctacggaacagctccagcccggatctcggtaccgtatgttggtatttcgaacgcctattcacacttttacgacggtttttccaaagtaccactgaaggaccagtcggcagcactaggtgactccctttatggtgcagcatctctaaatgacttcggtattttggctgttagagtagtcaatgatcacaacccgaccaaggtcacctccaaaatcagagtgtatctaaaacccaaacacatcagagtctggtgcccgcgtccaccgagggcagtggcgtactacggccctggagtggattacaaggatggtacgcttacacccctctccaccaaggatctgaccacatatggattcggacaccaaaacaaagcggtgtacactgcaggttacaaaatttgcaactaccacttggccactcaggatgatttgcaaaacgcagtgaacgtcatgtggagtagagacctcttagtcacagaatcaagagcccagggcaccgattcaatcgcaaggtgcaattgcaacgcaggggtgtactactgcgagtctagaaggaaatactacccagtatccttcgttggcccaacgttccagtacatggaggctaataactattacccagctaggtaccagtcccatatgctcattggccatggattcgcatctccaggggattgtggtggcatactcagatgtcaccacggggtgatagggatcattactgctggtggcgaagggttggttgcattttcagacattagagacttgtatgcctacgaagaagaagccatggaacaaggcatcaccaattacatagagtcacttggggccgcatttggaagtggatttactcagcagattagcgacaaaataacagagttgaccaatatggtgaccagtaccatcactgaaaagctacttaagaacttgatcaagatcatatcctcactagttattataactaggaactatgaagacaccacaacagtgctcgctaccctggcccttcttgggtgtgatgcttcaccatggcagtggcttagaaagaaagcatgcgatgttctggagataccttatgtcatcaagcaaggtgacagttggttgaagaagtttactgaagcatgcaacgcagctaagggactggagtgggtgtcaaacaaaatctcaaaattcattgattggctcaaggagaaaattatcccacaagctagagataagttggaatttgtaacaaaacttagacaactagaaatgctggaaaaccaaatctcaactatacaccaatcatgccctagtcaggaacaccaggaaattctattcaataatgtcagatggttatccatccagtctaagaggtttgcccctctttacgcagtggaagccaaaagaatacagaaactagagcatactattaacaactacatacagttcaagagcaaacaccgtattgaaccagtatgtttgctagtacatggcagccccggaacaggtaaatctgtagcaaccaacctgattgctagagccatagctgaaagagaaaacacgtccacgtactcgctacccccggatccatcacacttcgacggatacaaacaacagggagtggtgattatggacgacctgaatcaaaacccagatggtgcggacatgaagctgttctgtcagatggtatcaacagtggagtttataccacccatggcatccctggaggagaaaggaatcctgtttacttcaaattacgttctagcatccacaaactcaagcagaatttccccccccactgtggcacacagtgatgcattagccaggcgctttgcgttcgacatggacattcaggtcatgaatgagtattctagagatgggaaattgaacatggccatggctactgaaatgtgtaagaactgtcaccaaccagcaaactttaagagatgctgtcctttagtgtgtggtaaggcaattcaattaatggacaaatcttccagagttagatacagtattgaccagatcactacaatgattatcaatgagagaaacagaagatccaacattggcaattgtatggaggctttgtttcaaggaccactccagtataaagacttgaaaattgacatcaagacgagtccccctcctgaatgtatcaatgacttgctccaagcagttgactcccaggaggtgagagattactgtgagaagaagggttggatagtcaacatcaccagccaggttcaaacagaaaggaacatcaacagggcaatgacaattctacaagcggtgacaaccttcgccgcagtggctggagttgtctatgtcatgtataaactgtttgctggacaccagggagcatacactggtttaccaaacaaaaaacccaacgtgcccaccattcggacagcaaaggtacaaggaccagggttcgattacgcagtggctatggctaaaagaaacattgttacagcaactactagcaagggagagttcactatgttaggagtccacgacaacgtggctattttaccaacccacgcttcacctggtgaaagcattgtgatcgatggcaaagaagtggagatcttggatgccaaagcgctcgaagatcaagcaggaaccaatcttgaaatcactataatcactctaaagagaaatgaaaagttcagagacattagaccacatatacctactcaaatcactgagacaaatgatggagtcttgatcgtgaacactagcaagtaccccaatatgtatgttcctgtcggtgctgtgactgaacagggatatctaaatctcggtgggcgccaaactgctcgtactctaatgtacaactttccaaccagagcaggacagtgtggtggagtcatcacatgtactgggaaagtcatcgggatgcatgttggtgggaacggttcacacgggtttgcagcggccctgaagcgatcatacttcactcagagtcaaggtgaaatccagtggatgagaccttcgaaggaagtgggatatccaatcataaatgccccgtccaaaaccaagcttgaacccagtgctttccactatgtgtttgaaggggtgaaggaaccagcagtcctcactaaaaacgatcccaggcttaagacagactttgaggaggcaattttctccaagtacgtgggtaacaaaattactgaagtggatgagtacatgaaagaggcagtagaccactatgctggccagctcatgtcactagacatcaacacagaacaaatgtgcttggaggatgccatgtatggcactgatggtctagaagcacttgatttgtccaccagtgctggctacccttatgtagcaatgggaaagaagaagagagacatcttgaacaaacaaaccagagacactaaggaaatgcaaaaactgctcgacacatatggaatcaacctcccactggtgacttatgtaaaggatgaacttagatccaaaacaaaggttgagcaggggaaatccagattaattgaagcttctagtttgaatgactcagtggcaatgagaatggcttttgggaacctatatgctgcttttcacaaaaacccaggagtgataacaggttcagcagtggggtgcgatccagatttgttttggagcaaaattccggtattgatggaagagaagctgtttgcttttgactacacagggtatgatgcatctctcagccctgcttggttcgaggcactaaagatggtgcttgagaaaatcggattcggagacagagttgactacatcgactacctaaaccactcacaccacctgtacaagaataaaacatactgtgtcaagggcggtatgccatctggctgctcaggcacttcaatttttaactcaatgattaacaacttgattatcaggacactcttactgaaaacctacaagggcatagatttagaccacctaaaaatgattgcctatggtgatgatgtaattgcttcctacccccatgaagttgacgctagtctcctagcccaatcaggaaaagactatggactaactatgactccagctgacaaatcagctacatttgaaacagtcacatgggagaatgtaacattcttgaagagattcttcagggcagacgagaaatacccatttcttattcatccagtaatgccaatgaaggaaattcatgaatcaattagatggactaaagatcctaggaacactcaggatcacgttcgctctctgtgccttttagcttggcacaatggcgaagaagaatataacaaattcctagctaaaatcaggagtgtgccaattggaagagctttattgctcccagagtactcaacattgtaccgccgttggcttgactcattttagtaaccctacctcagtcgaattggattgggtcatactgttgtaggggtaaatttttctttaattcggag

SEQ ID NO: 6 - DNA, 627 bp, artificial
  IRES element from poliovirus 1 Mahoney
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggatt

SEQ ID NO: 7 - DNA, 12573 bp, bovine viral diarrhea virus
gtatacgagaattagaaaaggcactcgtatacgtattgggcaattaaaaataataattaggcctagggaacaaatccctctcagcgaaggccgaaaagaggctagccatgcccttagtaggactagcataatgaggggggtagcaacagtggtgagttcgttggatggcttaagccctgagtacagggtagtcgtcagtggttcgacgccttggaataaaggtctcgagatgccacgtggacgagggcatgcccaaagcacatcttaacctgagcgggggtcgcccaggtaaaagcagttttaaccgactgttacgaatacagcctgatagggtgctgcagaggcccactgtattgctactaaaaatctctgctgtacatggcacatggagttgatcacaaatgaacttttatacaaaacatacaaacaaaaacccgtcggggtggaggaacctgtttatgatcaggcaggtgatcccttatttggtgaaaggggagcagtccaccctcaatcgacgctaaagctcccacacaagagaggggaacgcgatgttccaaccaacttggcatccttaccaaaaagaggtgactgcaggtcgggtaatagcagaggacctgtgagcgggatctacctgaagccagggccactattttaccaggactataaaggtcccgtctatcacagggccccgctggagctctttgaggagggatccatgtgtgaaacgactaaacggatagggagagtaactggaagtgacggaaagctgtaccacatttatgtgtgtatagatggatgtataataataaaaagtgccacgagaagttaccaaagggtgttcaggtgggtccataataggcttgactgccctctatgggtcacaacttgctcagacacgaaagaagagggagcaacaaaaaagaaaacacagaaacccgacagactagaaagggggaaaatgaaaatagtgcccaaagaatctgaaaaagacagcaaaactaaacctccggatgctacaatagtggtggaaggagtcaaataccaggtgaggaagaagggaaaaaccaagagtaaaaacactcaggacggcttgtaccataacaaaaacaaacctcaggaatcacgcaagaaactggaaaaagcattgttggcgtgggcaataatagctatagttttgtttcaagttacaatgggagaaaacataacacagtggaacctacaagataatgggacggaagggatacaacgggcaatgttccaaaggggtgtgaatagaagtttacatggaatctggccagagaaaatctgtactggcgtcccttcccatctagccaccgatatagaactaaaaacaattcatggtatgatggatgcaagtgagaagaccaactacacgtgttgcagacttcaacgccatgagtggaacaagcatggttggtgcaactggtacaatattgaaccctggattctagtcatgaatagaacccaagccaatctcactgagggacaaccaccaagggagtgcgcagtcacttgtaggtatgatagggctagtgacttaaacgtggtaacacaagctagagatagccccacacccttaacaggttgcaagaaaggaaagaacttctcctttgcaggcatattgatgcggggcccctgcaactttgaaatagctgcaagtgatgtattattcaaagaacatgaacgcattagtatgttccaggataccactctttaccttgttgacgggttgaccaactccttagaaggtgccagacaaggaaccgctaaactgacaacctggttaggcaagcagctcgggatactaggaaaaaagttggaaaacaagagtaagacgtggtttggagcatacgctgcttccccttactgtgatgtcgatcgcaaaattggctacatatggtatacaaaaaattgcacccctgcctgcttacccaagaacacaaaaattgtcggccctgggaaatttggcaccaatgcagaggacggcaagatattacatgagatggggggtcacttgtcggaggtactactactttctttagtggtgctgtccgacttcgcaccggaaacagctagtgtaatgtacctaatcctacatttttccatcccacaaagtcacgttgatgtaatggattgtgataagacccagttgaacctcacagtggagctgacaacagctgaagtaataccagggtcggtctggaatctaggcaaatatgtatgtataagaccaaattggtggccttatgagacaactgtagtgttggcatttgaagaggtgagccaggtggtgaagttagtgttgagggcactcagagatttaacacgcatttggaacgctgcaacaactactgcttttttagtatgccttgttaagatagtcaggggccagatggtacagggcattctgtggctactattgataacaggggtacaagggcacttggattgcaaacctgaattctcgtatgccatagcaaaggacgaaagaattggtcaactgggggctgaaggccttaccaccacttggaaggaatactcacctggaatgaagctggaagacacaatggtcattgcttggtgcgaagatgggaagttaatgtacctccaaagatgcacgagagaaaccagatatctcgcaatcttgcatacaagagccttgccgaccagtgtggtattcaaaaaactctttgatgggcgaaagcaagaggatgtagtcgaaatgaacgacaactttgaatttggactctgcccatgtgatgccaaacccatagtaagagggaagttcaatacaacgctgctgaacggaccggccttccagatggtatgccccataggatggacagggactgtaagctgtacgtcattcaatatggacaccttagccacaactgtggtacggacatatagaaggtctaaaccattccctcataggcaaggctgtatcacccaaaagaatctgggggaggatctccataactgcatccttggaggaaattggacttgtgtgcctggagaccaactactatacaaagggggctctattgaatcttgcaagtggtgtggctatcaatttaaagagagtgagggactaccacactaccccattggcaagtgtaaattggagaacgagactggttacaggctagtagacagtacctcttgcaatagagaaggtgtggccatagtaccacaagggacattaaagtgcaagataggaaaaacaactgtacaggtcatagctatggataccaaactcggacctatgccttgcagaccatatgaaatcatatcaagtgaggggcctgtagaaaagacagcgtgtactttcaactacactaagacattaaaaaataagtattttgagcccagagacagctactttcagcaatacatgctaaaaggagagtatcaatactggtttgacctggaggtgactgaccatcaccgggattacttcgctgagtccatattagtggtggtagtagccctcttgggtggcagatatgtactttggttactggttacatacatggtcttatcagaacagaaggccttagggattcagtatggatcaggggaagtggtgatgatgggcaacttgctaacccataacaatattgaagtggtgacatacttcttgctgctgtacctactgctgagggaggagagcgtaaagaagtgggtcttactcttataccacatcttagtggtacacccaatcaaatctgtaattgtgatcctactgatgattggggatgtggtaaaggccgattcagggggccaagagtacttggggaaaatagacctctgttttacaacagtagtactaatcgtcataggtttaatcatagctaggcgtgacccaactatagtgccactggtaacaataatggcagcactgagggtcactgaactgacccaccagcctggagttgacatcgctgtggcggtcatgactataaccctactgatggttagctatgtgacagattattttagatataaaaaatggttacagtgcattctcagcctggtatctgcggtgttcttgataagaagcctaatatacctaggtagaatcgagatgccagaggtaactatcccaaactggagaccactaactttaatactattatatttgatctcaacaacaattgtaacgaggtggaaggttgacgtggctggcctattgttgcaatgtgtgcctatcttattgctggtcacaaccttgtgggccgacttcttaaccctaatactgatcctgcctacctatgaattggttaaattatactatctgaaaactgttaggactgatacagaaagaagttggctaggggggatagactatacaagagttgactccatctacgacgttgatgagagtggagagggcgtatatctttttccatcaaggcagaaagcacaggggaatttttctatactcttgccccttatcaaagcaacactgataagttgcgtcagcagtaaatggcagctaatatacatgagttacttaactttggactttatgtactacatgcacaggaaagttatagaagagatctcaggaggtaccaacataatatccaggttagtggcagcactcatagagctgaactggtccatggaagaagaggagagcaaaggcttaaagaagttttatctattgtctggaaggttgagaaacctaataataaaacataaggtaaggaatgagaccgtggcttcttggtacggggaggaggaagtctacggtatgccaaagatcatgactataatcaaggccagtacactgagtaagagcaggcactgcataatatgcactgtatgtgagggccgagagtggaaaggtggcacctgcccaaaatgtggacgccatgggaagccgataacgtgtgggatgtcgctagcagattttgaagaaagacactataaaagaatctttataagggaaggcaactttgagggtatgtgcagccgatgccagggaaagcataggaggtttgaaatggaccgggaacctaagagtgccagatactgtgctgagtgtaataggctgcatcctgctgaggaaggtgacttttgggcagagtcgagcatgttgggcctcaaaatcacctactttgcgctgatggatggaaaggtgtatgatatcacagagtgggctggatgccagcgtgtgggaatctccccagatacccacagagtcccttgtcacatctcatttggttcacggatgcctttcaggcaggaatacaatggctttgtacaatataccgctagggggcaactatttctgagaaacttgcccgtactggcaactaaagtaaaaatgctcatggtaggcaaccttggagaagaaattggtaatctggaacatcttgggtggatcctaagggggcctgccgtgtgtaagaagatcacagagcacgaaaaatgccacattaatatactggataaactaaccgcatttttcgggatcatgccaagggggactacacccagagccccggtgaggttccctacgagcttactaaaagtgaggaggggtctggagactgcctgggcttacacacaccaaggcgggataagttcagtcgaccatgtaaccgccggaaaagatctactggtctgtgacagcatgggacgaactagagtggtttgccaaagcaacaacaggttgaccgatgagacagagtatggcgtcaagactgactcagggtgcccagacggtgccagatgttatgtgttaaatccagaggccgttaacatatcaggatccaaaggggcagtcgttcacctccaaaagacaggtggagaattcacgtgtgtcaccgcatcaggcacaccggctttcttcgacctaaaaaacttgaaaggatggtcaggcttgcctatatttgaagcctccagcgggagggtggttggcagagtcaaagtagggaagaatgaagagtctaaacctacaaaaataatgagtggaatccagaccgtctcaaaaaacagagcagacctgaccgagatggtcaagaagataaccagcatgaacaggggagacttcaagcagattactttggcaacaggggcaggcaaaaccacagaactcccaaaagcagttatagaggagataggaagacacaagagagtattagttcttataccattaagggcagcggcagagtcagtctaccagtatatgagattgaaacacccaagcatctcttttaacctaaggataggggacatgaaagagggggacatggcaaccgggataacctatgcatcatacgggtacttctgccaaatgcctcaaccaaagctcagagctgctatggtagaatactcatacatattcttagatgaataccattgtgccactcctgaacaactggcaattatcgggaagatccacagattttcagagagtataagggttgtcgccatgactgccacgccagcagggtcggtgaccacaacaggtcaaaagcacccaatagaggaattcatagcccccgaggtaatgaaaggggaggatcttggtagtcagttccttgatatagcagggttaaaaataccagtggatgagatgaaaggcaatatgttggtttttgtaccaacgagaaacatggcagtagaggtagcaaagaagctaaaagctaagggctataactctggatactattacagtggagaggatccagccaatctgagagttgtgacatcacaatccccctatgtaatcgtggctacaaatgctattgaatcaggagtgacactaccagatttggacacggttatagacacggggttgaaatgtgaaaagagggtgagggtatcatcaaagatacccttcatcgtaacaggccttaagaggatggccgtgactgtgggtgagcaggcgcagcgtaggggcagagtaggtagagtgaaacccgggaggtattataggagccaggaaacagcaacagggtcaaaggactaccactatgacctcttgcaggcacaaagatacgggattgaggatggaatcaacgtgacgaaatcctttagggagatgaattacgattggagcctatacgaggaggacagcctactaataacccagctggaaatactaaataatctactcatctcagaagacttgccagccgctgttaagaacataatggccaggactgatcacccagagccaatccaacttgcatacaacagctatgaagtccaggtcccggtcctattcccaaaaataaggaatggagaagtcacagacacctacgaaaattactcgtttctaaatgccagaaagttaggggaggatgtgcccgtgtatatctacgctactgaagatgaggatctggcagttgacctcttagggctagactggcctgatcctgggaaccagcaggtagtggagactggtaaagcactgaagcaagtgaccgggttgtcctcggctgaaaatgccctactagtggctttatttgggtatgtgggttaccaggctctctcaaagaggcatgtcccaatgataacagacatatataccatcgaggaccagagactagaagacaccacccacctccagtatgcacccaacgccataaaaaccgatgggacagagactgaactgaaagaactggcgtcgggtgacgtggaaaaaatcatgggagccatttcagattatgcagctgggggactggagtttgttaaatcccaagcagaaaagataaaaacagctcctttgtttaaagaaaacgcagaagccgcaaaagggtatgtccaaaaattcattgactcattaattgaaaataaagaagaaataatcagatatggtttgtggggaacacacacagcactatacaaaagcatagctgcaagactggggcatgaaacagcgtttgccacactagtgttaaagtggctagcttttggaggggaatcagtgtcagaccacgtcaagcaggcggcagttgatttagtggtctattatgtgatgaataagccttccttcccaggtgactccgagacacagcaagaagggaggcgattcgtcgcaagcctgttcatctccgcactggcaacctacacatacaaaacttggaattaccacaatctctctaaagtggtggaaccagccctggcttacctcccctatgctaccagcgcattaaaaatgttcaccccaacgcggctggagagcgtggtgatactgagcaccacgatatataaaacatacctctctataaggaaggggaagagtgatggattgctgggtacggggataagtgcagccatggaaatcctgtcacaaaacccagtatcggtaggtatatctgtgatgttgggggtaggggcaatcgctgcgcacaacgctattgagtccagtgaacagaaaaggaccctacttatgaaggtgtttgtaaagaacttcttggatcaggctgcaacagatgagctggtaaaagaaaacccagaaaaaattataatggccttatttgaagcagtccagacaattggtaaccccctgagactaatataccacctgtatggggtttactacaaaggttgggaggccaaggaactatctgagaggacagcaggcagaaacttattcacattgataatgtttgaagccttcgagttattagggatggactcacaagggaaaataaggaacctgtccggaaattacattttggatttgatatacggcctacacaagcaaatcaacagagggctgaagaaaatggtactggggtgggcccctgcaccctttagttgtgactggacccctagtgacgagaggatcagattgccaacagacaactatttgagggtagaaaccaggtgcccatgtggctatgagatgaaagctttcaaaaatgtaggtggcaaacttaccaaagtggaggagagcgggcctttcctatgtagaaacagacctggtaggggaccagtcaactacagagtcaccaagtattacgatgacaacctcagagagataaaaccagtagcaaagttggaaggacaggtagagcactactacaaaggggtcacagcaaaaattgactacagtaaaggaaaaatgctcttggccactgacaagtgggaggtggaacatggtgtcataaccaggttagctaagagatatactggggtcgggttcaatggtgcatacttaggtgacgagcccaatcaccgtgctctagtggagagggactgtgcaactataaccaaaaacacagtacagtttctaaaaatgaagaaggggtgtgcgttcacctatgacctgaccatctccaatctgaccaggctcatcgaactagtacacaggaacaatcttgaagagaaggaaatacccaccgctacggtcaccacatggctagcttacaccttcgtgaatgaagacgtagggactataaaaccagtactaggagagagagtaatccccgaccctgtagttgatatcaatttacaaccagaggtgcaagtggacacgtcagaggttgggatcacaataattggaagggaaaccctgatgacaacgggagtgacacctgtcttggaaaaagtagagcctgacgccagcgacaaccaaaactcggtgaagatcgggttggatgagggtaattacccagggcctggaatacagacacatacactaacagaagaaatacacaacagggatgcgaggcccttcatcatgatcctgggctcaaggaattccatatcaaatagggcaaagactgctagaaatataaatctgtacacaggaaatgaccccagggaaatacgagacttgatggctgcagggcgcatgttagtagtagcactgagggatgtcgaccctgagctgtctgaaatggtcgatttcaaggggacttttttagatagggaggccctggaggctctaagtctcgggcaacctaaaccgaagcaggttaccaaggaagctgttaggaatttgatagaacagaaaaaagatgtggagatccctaactggtttgcatcagatgacccagtatttctggaagtggccttaaaaaatgataagtactacttagtaggagatgttggagagctaaaagatcaagctaaagcacttggggccacggatcagacaagaattataaaggaggtaggctcaaggacgtatgccatgaagctatctagctggttcctcaaggcatcaaacaaacagatgagtttaactccactgtttgaggaattgttgctacggtgcccacctgcaactaagagcaataaggggcacatggcatcagcttaccaattggcacagggtaactgggagcccctcggttgcggggtgcacctaggtacaataccagccagaagggtgaagatacacccatatgaagcttacctgaagttgaaagatttcatagaagaagaagagaagaaacctagggttaaggatacagtaataagagagcacaacaaatggatacttaaaaaaataaggtttcaaggaaacctcaacaccaagaaaatgctcaacccagggaaactatctgaacagttggacagggaggggcgcaagaggaacatctacaaccaccagattggtactataatgtcaagtgcaggcataaggctggagaaattgccaatagtgagggcccaaaccgacaccaaaacctttcatgaggcaataagagataagatagacaagagtgaaaaccggcaaaatccagaattgcacaacaaattgttggagattttccacacgatagcccaacccaccctgaaacacacctacggtgaggtgacgtgggagcaacttgaggcgggggtaaatagaaagggggcagcaggcttcctggagaagaagaacatcggagaagtattggattcagaaaagcacctggtagaacaattggtcagggatctgaaggccgggagaaagataaaatattatgaaactgcaataccaaaaaatgagaagagagatgtcagtgatgactggcaggcaggggacctggtggttgagaagaggccaagagttatccaataccctgaagccaagacaaggctagccatcactaaggtcatgtataactgggtgaaacagcagcccgttgtgattccaggatatgaaggaaagacccccttgttcaacatctttgataaagtgagaaaggaatgggactcgttcaatgagccagtggccgtaagttttgacaccaaagcctgggacactcaagtgactagtaaggatctgcaacttattggagaaatccagaaatattactataagaaggagtggcacaagttcattgacaccatcaccgaccacatgacagaagtaccagttataacagcagatggtgaagtatatataagaaatgggcagagagggagcggccagccagacacaagtgctggcaacagcatgttaaatgtcctgacaatgatgtacggcttctgcgaaagcacaggggtaccgtacaagagtttcaacagggtggcaaggatccacgtctgtggggatgatggcttcttaataactgaaaaagggttagggctgaaatttgctaacaaagggatgcagattcttcatgaagcaggcaaacctcagaagataacggaaggggaaaagatgaaagttgcctatagatttgaggatatagagttctgttctcataccccagtccctgttaggtggtccgacaacaccagtagtcacatggccgggagagacaccgctgtgatactatcaaagatggcaacaagattggattcaagtggagagaggggtaccacagcatatgaaaaagcggtagccttcagtttcttgctgatgtattcctggaacccgcttgttaggaggatttgcctgttggtcctttcgcaacagccagagacagacccatcaaaacatgccacttattattacaaaggtgatccaataggggcctataaagatgtaataggtcggaatctaagtgaactgaagagaacaggctttgagaaattggcaaatctaaacctaagcctgtccacgttgggggtctggactaagcacacaagcaaaagaataattcaggactgtgttgccattgggaaagaagagggcaactggctagttaagcccgacaggctgatatccagcaaaactggccacttatacatacctgataaaggctttacattacaaggaaagcattatgagcaactgcagctaagaacagagacaaacccggtcatgggggttgggactgagagatacaagttaggtcccatagtcaatctgctgctgagaaggttgaaaattctgctcatgacggccgtcggcgtcagcagctgagacaaaatgtatatattgtaaataaattaatccatgtacatagtgtatataaatatagttgggaccgtccacctcaagaagacgacacgcccaacacgcacagctaaacagtagtcaagattatctacctcaagataacactacatttaatgcacacagcactttagctgtatgaggatacgcccgacgtctatagttggactagggaagacctctaacag

SEQ ID NO: 8 - DNA, 900 bp, artificial
  IRES element from bovine viral diarrhea virus
gtatacgagaattagaaaaggcactcgtatacgtattgggcaattaaaaataataattaggcctagggaacaaatccctctcagcgaaggccgaaaagaggctagccatgcccttagtaggactagcataatgaggggggtagcaacagtggtgagttcgttggatggcttaagccctgagtacagggtagtcgtcagtggttcgacgccttggaataaaggtctcgagatgccacgtggacgagggcatgcccaaagcacatcttaacctgagcgggggtcgcccaggtaaaagcagttttaaccgactgttacgaatacagcctgatagggtgctgcagaggcccactgtattgctactaaaaatctctgctgtacatggcacatggagttgatcacaaatgaacttttatacaaaacatacaaacaaaaacccgtcggggtggaggaacctgtttatgatcaggcaggtgatcccttatttggtgaaaggggagcagtccaccctcaatcgacgctaaagctcccacacaagagaggggaacgcgatgttccaaccaacttggcatccttaccaaaaagaggtgactgcaggtcgggtaatagcagaggacctgtgagcgggatctacctgaagccagggccactattttaccaggactataaaggtcccgtctatcacagggccccgctggagctctttgaggagggatccatgtgtgaaacgactaaacggatagggagagtaactggaagtgacggaaagctgtaccacatttatgtgtgtatagatggatgtataataataaaaagtgccacgagaagttaccaaagggtgttcaggtgggtccataataggcttgactgccctctatgggtcacaacttgctcagacacgaa

(19) United States
(12) Patent Application Publication
(10) Pub. No.: US 2011/0014624 A1
(43) Pub. Date: Jan. 20, 2011

(54) Methods For Identifying Cells Suitable For Large-Scale Production of Recombinant Proteins

(75) Inventors: Kevin Michael McCarthy, Haverhill, MA (US); Megan Jill Hone, Hudson, NH (US); Robin Allison Heller-Harrison, Andover, MA (US); Mark William Leonard, Manchester, NH (US)

Correspondence Address: WYETH LLC;PATENT LAW GROUP, 5 GIRALDA FARMS, MADISON, NJ, 07940, US

(73) Assignee: Wyeth LLC, Madison, NJ (US)

(21) Appl. No.: 12/921,717

(22) PCT Filed: Mar. 12, 2009
(86) PCT No.: PCT/US09/36962

§ 371 Date: Sep. 9, 2010

Related U.S. Application Data
(60) Provisional application No. 61/035,804, filed on Mar. 12, 2008.

(51) Int. Cl. C12Q 1/68; C12N 5/071

(52) U.S. Cl. 435/6; 435/325

(57) ABSTRACT

The present invention provides methods of identifying a clonal population of cells suitable for large-scale production of a protein of interest. The invention further provides methods for high-throughput screening for genetic rearrangements in the gene encoding the protein of interest, whereby the absence of a deletion in the gene encoding the protein of interest indicates that the cell is suitable for large-scale production of the protein of interest.

### FIELD

The present invention relates generally to gene expression and protein production and, more specifically, to methods for identifying cells suitable for use in large-scale production of recombinant proteins.

### BACKGROUND

One of the major goals in the biotechnology industry is the development of stable cell-lines suitable for the expression and large-scale production of recombinant proteins, such as, but not limited to, recombinant antibodies. Standard methodologies require time consuming and labor intensive development of suitable recombinant host cell-lines that express the gene of interest. Conventionally, cells are transfected by an expression vector containing the gene of interest and a selectable marker gene. The entire population of cells then undergoes a process of selection to remove cells that failed to take up the expression vector. The vector containing pool is then, typically, subcloned and screened for high-level expression. Each of the resulting high-level expressing clones is then expanded and further adapted to growth in culture. However, many times these cells are unstable and it is often the case that there is a loss of expression of the recombinant protein and/or polypeptide.

The instability of gene expression hinders the development of production cell lines for protein therapeutics. Loss of expression can occur through a variety of mechanisms including, but not limited to DNA methylation, homologous recombination and non-homologous recombination. Any of these mechanisms may lead to loss of all or part of the expression cassette, possibly leaving the cell susceptible to the selecting agent.

There remains a need in the art for improved methods for identifying a clonal population of cells suitable for expression and large-scale production of recombinant proteins and/or polypeptides including, but not limited to, antibody heavy and light chains. The present invention addresses these needs and provides a method for identifying a clonal population of recombinant cells that stably expresses a protein of interest and which is suitable for large-scale production of a recombinant protein/polypeptide.

The citation of any reference herein should not be deemed as an admission that such reference is available as prior art to the instant invention.

### SUMMARY

One aspect of the invention provides a method of identifying a clonal population of cells suitable for large-scale production of a protein of interest, the method comprising:
a) transfecting a population of cells with a nucleic acid construct comprising a gene encoding a protein of interest;
b) isolating a clonal population of cells expressing the gene encoding the protein of interest;
c) determining the presence or absence of a rearrangement of the gene encoding the protein of interest in the clonal population;
d) selecting a clonal population of cells from step c) that lack the rearrangement of the gene encoding the protein of interest; and
e) culturing the clonal population of cells from step d) for large-scale production of the protein of interest.

In one embodiment, the invention provides a method of identifying a clonal population of cells suitable for large-scale production of a protein of interest, the method comprising:
a) transfecting a population of cells with a nucleic acid construct comprising in sequential order a coding region for a tripartite leader sequence (TPL), an intron, a gene encoding a protein of interest, an Internal Ribosome Entry Site (IRES) and a coding region for a selectable marker;
b) isolating a clonal population of cells expressing the gene encoding the protein of interest and the selectable marker;
c) determining the presence or absence of a rearrangement of the gene encoding the protein of interest in the clonal population;
d) selecting a clonal population of cells from step c) that lack the rearrangement of the gene encoding the protein of interest; and
e) culturing the clonal population of cells from step d) for large-scale production of the protein of interest.

In one embodiment, the method further comprises isolating and purifying the protein of interest from the clonal population of cells.

In one embodiment, the large-scale production of step e) above comprises culturing the cells in a volume of greater than two liters of cell culture medium.

In one embodiment, the method provides for a nucleic acid construct comprising an intron sequence located 5′ to the gene encoding the protein of interest.

In one embodiment, the method provides for a nucleic acid construct comprising a coding region for a tripartite leader (TPL) sequence located 5′ to the intron sequence.

In one embodiment, the method provides for a nucleic acid construct further comprising, in sequential order, an IRES operably linked to a coding region for a selectable marker, wherein the IRES and selectable marker coding region are located 3′ to the gene encoding the protein of interest.

In one embodiment, the method provides for a nucleic acid construct wherein the gene encodes a heavy chain of an immunoglobulin molecule.

In one embodiment, the method provides for a nucleic acid construct wherein the gene encodes a light chain of an immunoglobulin molecule.

In one embodiment, the method provides for a nucleic acid construct wherein the selectable marker is selected from the group consisting of dihydrofolate reductase (DHFR), neomycin transferase, histidinol, hygromycin, glutamine synthetase, zeocin and phleomycin.

In one embodiment, the method provides for a nucleic acid construct wherein the IRES is selected from the group consisting of SEQ ID NOs: 4, 6 and 8.

In one embodiment, the method provides for a nucleic acid construct wherein the rearrangement comprises a deletion of all or part of the gene encoding the protein of interest.

In one embodiment, the method provides for a nucleic acid construct wherein the deletion in the gene encoding the protein of interest is detected in a nucleic acid selected from the group consisting of DNA, pre-mRNA and mRNA.

In one embodiment, the method provides for a nucleic acid construct wherein the gene encodes a protein of interest, which is an antibody, a fusion protein, or a small modular immunopharmaceutical product (SMIP).

In one embodiment, the method provides for a nucleic acid construct wherein the gene encodes a therapeutic antibody.

In one embodiment, the method provides for a determining step which comprises helicase dependent amplification or any polymerase chain reaction (PCR) selected from the group consisting of RT-PCR, inverse PCR, quantitative PCR, real-time PCR, and in situ PCR.

A second aspect of the invention provides an assay for identifying a clonal population of cells suitable for large-scale production of a protein of interest, comprising:
a) culturing cells comprising a nucleic acid construct comprising a gene encoding a protein of interest to produce a clonal population of cells;
b) amplifying by polymerase chain reaction (PCR) a portion of the gene, wherein the amplification is carried out using a first primer and a second primer, wherein the first primer hybridizes to a nucleotide sequence that is 5′ to the gene, and the second primer hybridizes to a nucleotide sequence that is 3′ to the gene; and
c) determining the presence or absence of a deletion of all or part of the gene in the amplified portion of the gene;
   wherein the absence of the deletion identifies the clonal population of cells as suitable for large-scale production of the protein of interest.

In one embodiment, the assay provides a nucleic acid construct which further comprises a coding region for a tripartite leader (TPL) sequence located 5′ to the gene encoding the protein of interest.

In one embodiment, the assay provides a nucleic acid construct, which further comprises, in sequential order, an Internal Ribosome Entry Site (IRES) operably linked to a coding region for a selectable marker, wherein the IRES and the selectable marker coding region are located 3′ to the gene encoding the protein of interest.

In one embodiment, the assay provides an amplifying step which comprises hybridizing the first primer to the coding region for the TPL sequence, and hybridizing the second primer to the coding region for the selectable marker.

A third aspect of the invention provides a clonal population of cells suitable for large scale production of a protein of interest produced by any of the methods described above.

### BRIEF DESCRIPTION OF THE DRAWINGS

FIG. 1. Depiction of Several Possible Vector Combinations for Co-Expression of a Protein and a Selectable Marker

(A) Expression of a heavy chain gene with a single selectable marker in the absence of an internal ribosomal entry site (IRES). Expression of the gene product is driven by a promoter and the transcript stabilized by a polyadenylation sequence (pA). The unlabeled box in the lower figure that lies between the pA and the promoter 2 denotes intervening DNA.

(B) Expression of a light chain gene with a single selectable marker in the absence of an IRES. Expression of the gene product is driven by a promoter and the transcript stabilized by a polyadenylation sequence (pA). The unlabeled box in the lower figure that lies between the pA and the promoter 2 denotes intervening DNA.

(C) Expression of a heavy chain or light chain gene using a bicistronic message. Each mRNA has an IRES to allow translation of a second gene product from the same transcript, in this case the second gene product is a selectable marker.

FIG. 2. Northern Blot Analysis of Antibody Heavy Chain-DHFR Bicistronic Transcript Loss Over Time

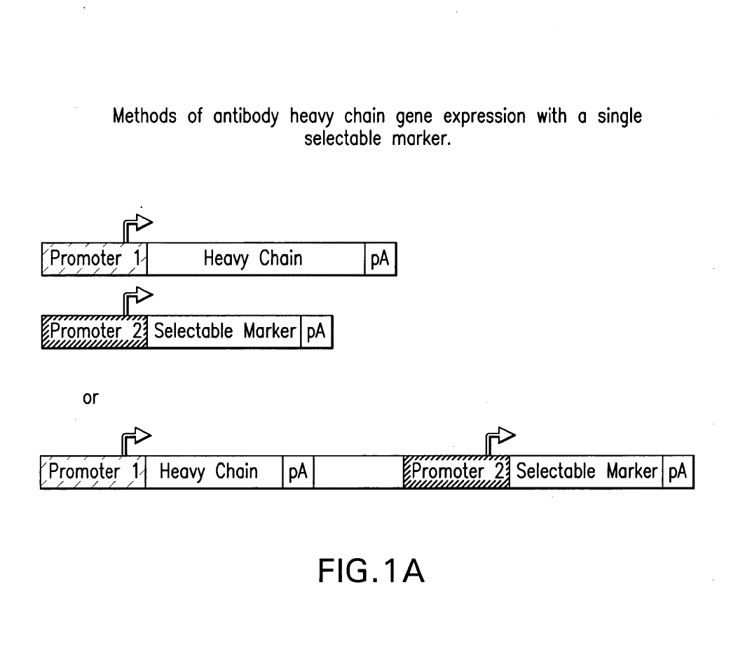

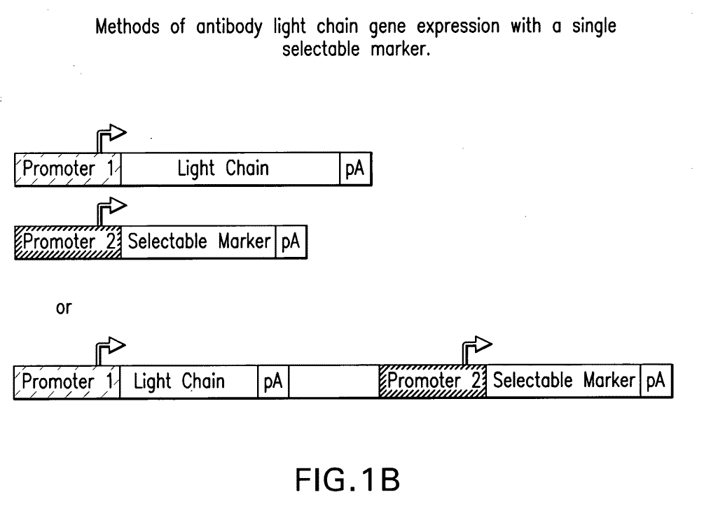

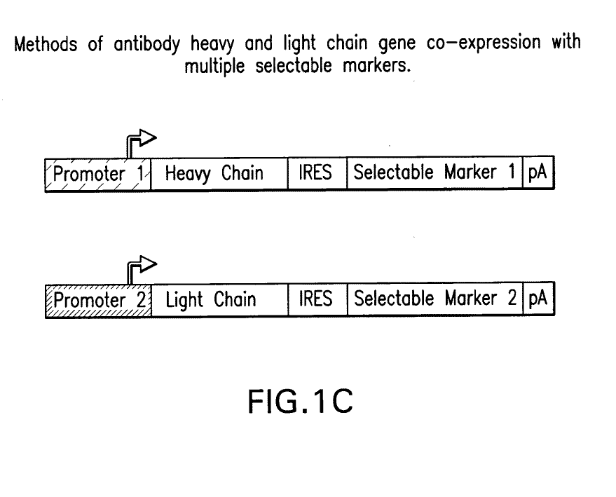

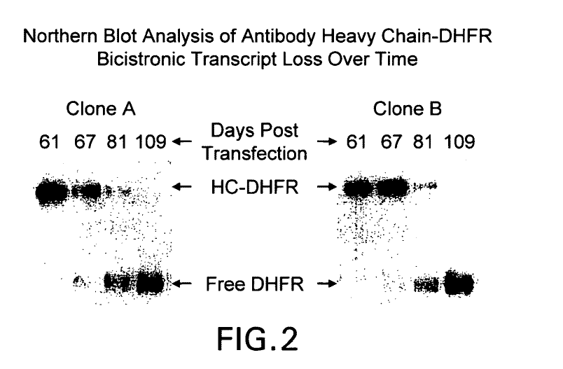

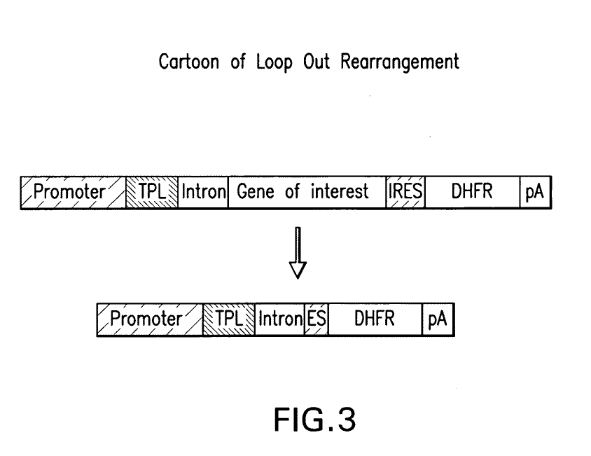

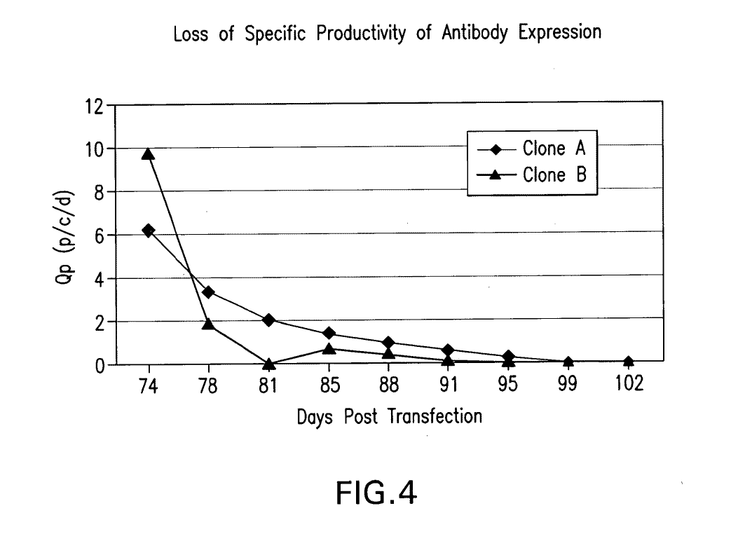

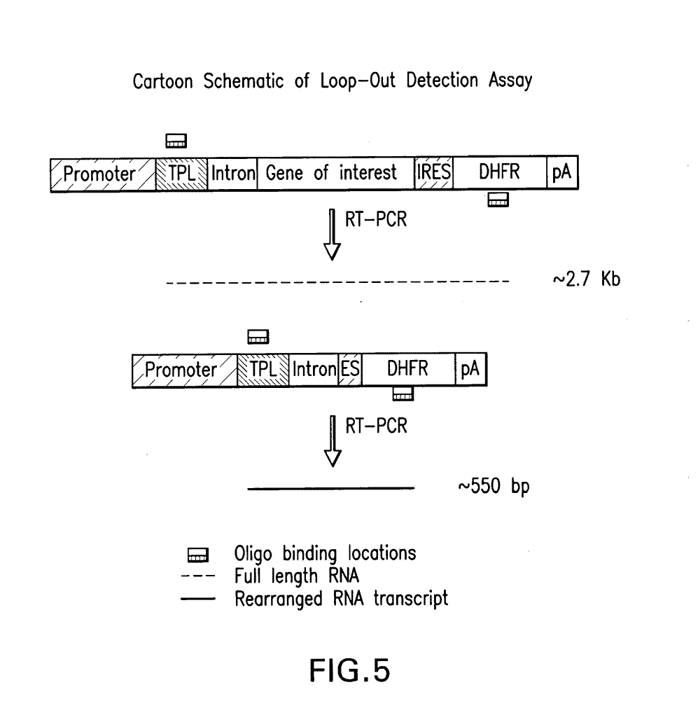

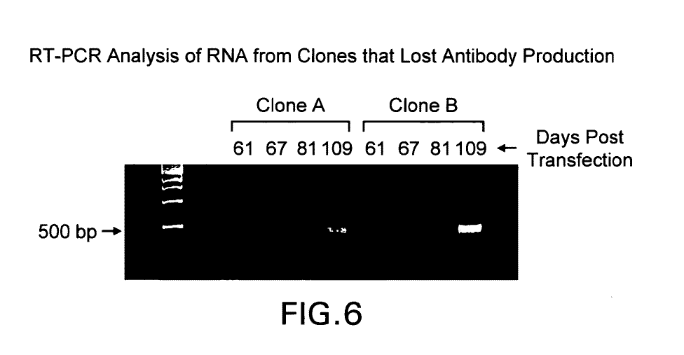

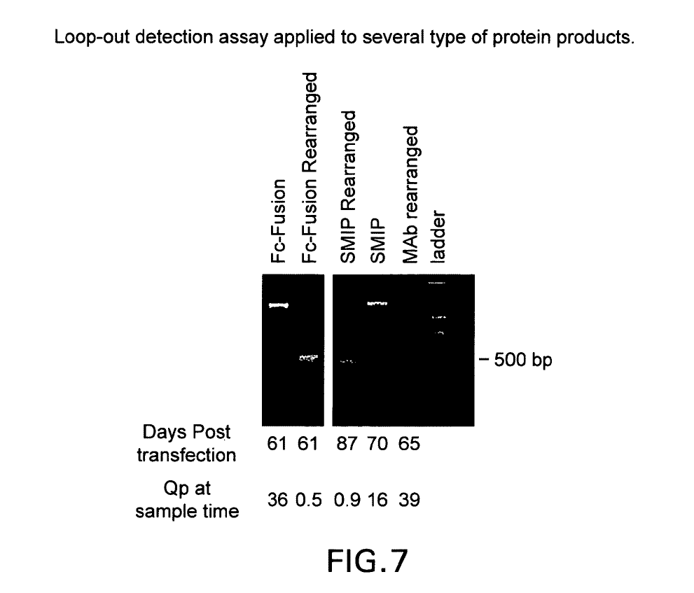

Cells were transfected with heavy chain DNA linked to DHFR via an IRES as depicted in FIG. 1C. Cells were selected for DHFR expression in chemically defined media containing methotrexate, expanded and adapted to serum free suspension. At the time points indicated for clones A and B expressing an antibody, RNA was isolated and 2 ug was separated on a 1.2% agarose gel. The nucleic acids were transferred to a nylon membrane and UV crosslinked. A ³²P labeled probe was generated by random prime reaction to DNA corresponding to the full length cDNA for mouse DHFR. This probe was allowed to hybridize to the membrane overnight at 42° C. and subsequently washed in 2×SSC/0.1% SDS at room temperature and then 0.1×SSC/0.1% SDS at 65° C. The membrane was exposed to X-ray film and developed. Shown is the transcript corresponding to nucleic acid complementary to the probe sequence. The full length bicistronic message is designated as HC-DHFR. The rearranged, smaller transcript from a genetic rearrangement is noted as Free DHFR.

FIG. 3: Schematic of Loop Out Rearrangement

Schematic of the rearranged Free DHFR transcript shown in FIG. 2. Cloning and sequencing of a few RNA products demonstrated that the resultant RNA corresponding to the Free DHFR was a genetic rearrangement between the lead intron and the IRES.

FIG. 4: Loss of Specific Productivity of Antibody Expression

Specific productivity of antibody production by clones A and B corresponds to loss of HC-DHFR transcript and onset of Free DHFR transcript. Media was collected from cell lines A and B and specific antibody quantity was measured. Productivity was normalized to pg of protein produced per cell per day (p/c/d). By day 95 post-transfection, the quantity of protein production is almost eliminated.

FIG. 5: Schematic of Loop-Out Detection Assay

Schematic of the loop-out detection assay. The primer annealing points are designated by the boxes positioned above and below the vector as shown in the figure. The forward primer anneals in the tripartite leader (TPL) and the reverse primer anneals in the mouse DHFR gene (DHFR). A full length RT-PCR product of an intact construct containing a typical antibody heavy chain is ˜2.7 Kb. If the gene of interest (in this case a heavy chain gene) is excised, the resulting RT-PCR product using the same primers is about 550 bp.

FIG. 6: RT-PCR Analysis of RNA from Clones that Lost Antibody Production

The loop-out detection assay is applied to RNA samples corresponding to clones A and B in FIGS. 2 and 4. RNA isolated from the earliest time point (day 61) demonstrates a detectable rearranged gene product in clone A. A similar sized RT-PCR product is detectable at day 67 in clone B. Both products are detected earlier than the corresponding northern blot demonstrating the sensitivity of the assay and much earlier than the first media sample to be analyzed (day 74 in FIG. 4).

FIG. 7: Loop-Out Detection Assay (LODA) Applied to Several Classes of Protein Products.

The Loop-out detection assay is applied to clonal populations of cells expressing an Fc-fusion protein, a Small Modular Immunopharmaceutical (SMIP) or an antibody. Cells were transfected and cultured as before. LODA was applied to cells that had already lost productivity at the time of RNA sampling (Qps of 0.5 and 0.9 respectively) or to an antibody (Qp of 39), which will lose expression similar to MAb clones A and B in FIG. 6. Qp refers to the specific productivity of a cell. It is the measurement of the amount of protein a single cell will make in a twenty-four (24) hour period (picograms/cell/day).

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure.

Accordingly, in the present application, there may be employed conventional molecular biology, microbiology, and recombinant DNA techniques within the skill of the art. Such techniques are explained fully in the literature. See, e.g., Byrd, C M and Hruby, D E, Methods in Molecular Biology, Vol. 269: Vaccinia Virus and Poxvirology, Chapter 3, pages 31-40; Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Although any methods and materials similar or equivalent to those described herein can be used in the practice or testing of the invention, the preferred methods and materials are now described. All publications mentioned herein are incorporated herein by reference in their entirety.

### Definitions

The terms used herein have the meanings recognized and known to those of skill in the art, however, for convenience and completeness, particular terms and their meanings are set forth below.

The term “about” means within 20%, more preferably within 10% and more preferably within 5%.

As used herein, “amplifying” refers to the generation of additional copies of a nucleic acid sequence. A variety of methods have been developed to amplify nucleic acid sequences, including the polymerase chain reaction (PCR). PCR amplification of a nucleic acid sequence generally results in the exponential amplification of a nucleic acid sequence(s).

The invention encompasses proteins including antibodies and other antigen-binding proteins. For purposes of this application, the term “antibody” is meant to include any antigen-binding protein described herein.

The term “antibody” includes a protein comprising at least one, and typically two, VH domains or portions thereof, and/or at least one, and typically two, VL domains or portions thereof. In certain embodiments, the antibody is a tetramer of two heavy immunoglobulin chains and two light immunoglobulin chains, wherein the heavy and light immunoglobulin chains are inter-connected by, e.g., disulfide bonds. The antibodies, or a portion thereof, can be obtained from any origin, including, but not limited to, rodent, primate (e.g., human and non-human primate), camelid, shark as well as recombinantly produced, e.g., chimeric, humanized, and/or in vitro generated, e.g., by methods well known to those of skill in the art.

This invention also encompasses “antigen-binding fragments of antibodies”, which include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; (vi) a camelid or camelized variable domain, e.g., a VHH domain; (vii) a single chain Fv (scFv); (viii) a bispecific antibody; and (ix) one or more antigen binding fragments of an immunoglobulin fused to an Fc region. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-26; Huston et al. (1988) Proc. Natl. Acad. Sci. U.S.A. 85:5879-83). Such single chain antibodies are also intended to be encompassed within the term “antigen-binding fragment” of an antibody. These antibody fragments are obtained using conventional techniques known to those skilled in the art, and the fragments are evaluated for function in the same manner as are intact antibodies.

The invention also encompasses single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, cow and shark. According to one aspect of the invention, a single domain antibody as used herein is a naturally occurring single domain antibody known as heavy chain antibody devoid of light chains. Such single domain antibodies are disclosed in WO 9404678 for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention. Single domain antibodies also include shark IgNARs; see, e.g., Dooley et al., Proc. Natl. Acad. Sci. U.S.A., 103:1846-1851 (2006).

Other than “bispecific” or “bifunctional” antibodies, an antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

In embodiments where the protein is an antibody or a fragment thereof, it can include at least one, or two full-length heavy chains, and at least one, or two light chains. Alternatively, the antibodies or fragments thereof can include only an antigen-binding fragment (e.g., an Fab, F(ab′)₂, Fv or a single chain Fv fragment). The antibody or fragment thereof can be a monoclonal or single specificity antibody. The antibody or fragment thereof can also be a human, humanized, chimeric. CDR-grafted, or in vitro generated antibody. In yet other embodiments, the antibody has a heavy chain constant region chosen from, e.g., IgG1, IgG2, IgG3, or IgG4. In another embodiment, the antibody has a light chain chosen from, e.g., kappa or lambda. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). Typically, the antibody or fragment thereof specifically binds to a predetermined antigen, e.g., an antigen associated with a disorder, e.g., a neurodegenerative, metabolic, inflammatory, autoimmune and/or a malignant disorder.

Proteins described herein, optionally, further include a moiety that enhances one or more of, e.g., stability, effector cell function or complement fixation. For example, an antibody or antigen-binding protein can further include a pegylated moiety, albumin, or a heavy and/or a light chain constant region.

A “therapeutic antibody” relates to any of the above antibody molecules, either alone or coupled to a moiety that allows for targeting to a particular receptor or cell type, or to the site of injury, or coupled to a chemical or protein moiety that allows for enhanced uptake by a cell, whereby such therapeutic antibody is used to treat a disease or to ameliorate at least one symptom associated with the disease.

Antibodies are generally made, for example, via traditional hybridoma techniques (Kohler et al., Nature 256:495-499 (1975)), recombinant DNA methods (U.S. Pat. No. 4,816,567), or phage display techniques using antibody libraries (Clackson et al., Nature 352:624-628 (1991); Marks et al., J. Mol. Biol. 222:581-597 (1991)). For various other antibody production techniques, see Antibodies: A Laboratory Manual, eds. Harlow et al., Cold Spring Harbor Laboratory, 1988.

Further, the antibodies may be tagged with a detectable or functional label. These labels include radiolabels (e.g., ¹³¹I or ⁹⁹Tc), enzymatic labels (e.g., horseradish peroxidase or alkaline phosphatase), and other chemical moieties (e.g., biotin).

The terms “cell”, or “cells”, or “host cell” and the like, as used herein, is intended to include any individual cell or cell culture (a “population of cells”), which can be or have been recipients for vectors or the incorporation of exogenous nucleic acid molecules, polynucleotides and/or proteins. The terms “cell”, or “cells”, may include the progeny of a single cell; however, the progeny may not necessarily be completely identical (in morphology or in genomic or total DNA complement) to the original parent cell due to natural, accidental, or deliberate mutation. The cells may be prokaryotic or eukaryotic, and may include, but are not limited to, bacterial cells, mammalian cells, animal cells (e.g., hamster, murine, rat, simian or human), insect cells and yeast cells.

A “clonal population of cells”, as used herein, are different from the cells noted above in that the term generally refers to a population of cells that originated from a single isolated cell.

A “coding region” or “coding sequence” or a sequence which “encodes” a selected polypeptide, is a nucleic acid molecule which is transcribed (in the case of DNA) and translated (in the case of mRNA) into a polypeptide when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, genomic DNA, cDNA, and mRNA sequences of viral, prokaryotic, eukaryotic and synthetic origin. A transcription termination sequence may be located 3′ to the coding sequence.

The sequence for mouse “Dihydrofolate Reductase” or “DHFR” is shown in Genbank sequence: L26316.

A “fusion molecule” is a protein containing two or, more operably associated, e.g., linked, moieties, e.g., protein moieties. Preferably, the moieties are covalently associated. The moieties can be directly associated, or connected via a spacer or linker.

“Gene expression” refers to the conversion of the information, contained in a gene, into a gene product. A gene product can be the direct transcriptional product of a gene (e.g., pro-mRNA, mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, 5 myristilation, and glycosylation.

“Helicase Dependent Amplification” (HAD) is a method for in vitro DNA amplification much like polymerase chain reaction (PCR). PCR involves denaturation of the double-stranded DNA with heat into single strands and copying the single strands to create new double-stranded DNA. Instead of these thermocycles, HDA mimics nature's method of replicating DNA by using the enzyme helicase to denature the DNA at a constant temperature of 37° C.

A nucleic acid that “hybridizes” (anneals) to the nucleic acid of the present invention may do so under “conditions of low stringency”. By way of example and not limitation, procedures using such conditions of low stringency are as follows (see also Shilo and Weinberg, 1981, Proc. Natl. Acad. Sci. U.S.A. 78, 6789 6792). Filters containing DNA are pretreated for 1 h at 42° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 42° C., and then washed for 30 min at 25° C. in a solution containing 2×SSC, 25 mM Tris-HCl (pH 7.4), 5 mM EDTA, and 0.1% SDS. The wash solution is replaced with fresh solution and incubated an additional 1 h at 65° C. Filters are blotted dry and exposed for autoradiography. If necessary, filters are washed for a third time at 65-68° C. and re-exposed to film. Other conditions of low stringency that may be used are well known in the art (e.g., as employed for cross-species hybridizations).

A nucleic acid that “hybridizes” (anneals) to the nucleic acid of the present invention may do so under “conditions of high stringency”. By way of example and not limitation, procedures using such conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5 20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency that may be used are well known in the art.

A nucleic acid that “hybridizes” (anneals) to the nucleic acid of the present invention may do so under “conditions of moderate stringency”. For example, but not limited to, procedures using such conditions of moderate stringency are as follows: filters comprising immobilized DNA are pretreated for 6 hours at 55° C. in a solution containing 6×SSC, 5×Denhardt's solution, 0.5% SDS and 100 μg/ml denatured salmon sperm DNA. Hybridizations are carried out in the same solution with 5-20×10⁶ cpm ³²P-labeled probe. Filters are incubated in hybridization mixture for 18-20 hours at 55° C., and then washed twice for 30 minutes at 60° C. in a solution containing 1×SSC and 0.1% SDS. Filters are blotted dry and exposed for autoradiography. Washing of filters is done at 37° C. for 1 hour in a solution containing 2×SSC, 0.1% SDS. Other conditions of moderate stringency that may be used are well known in the art. (see, e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987 1997 Current Protocols, COPYRGT. 1994 1997 John Wiley and Sons, Inc.).

In general terms, the word “isolating” refers to the removal of a material of interest from its original environment (e.g., a natural environment if it is naturally occurring, or from an environment into which it has been placed). For example, an “isolated” peptide or protein is substantially free of cellular material or other contaminating proteins from the cell or tissue source from which the protein is derived, or substantially free of chemical precursors or other chemicals when chemically synthesized. In the present invention, the method described provides for a means of “isolating” a clonal population of cells expressing a protein of interest from other cells not expressing the protein of interest.

A “leader sequence” is a sequence at the 5′ end of an mRNA that is not translated into protein. It is the length of untranslated mRNA from the 5′ end to the initiation codon AUG.″ A “tripartite leader (TPL) sequence” is described in: Zhang Y, Dolph P J, Schneider R J. Secondary structure analysis of adenovirus tripartite leader. J Biol Chem. 1989 Jun. 25; 264(18):10679-84.

The term “nucleic acid molecule” or “nucleic acid sequence” refers to both double- and single-stranded nucleotide sequences and refers to, but is not limited to, genomic DNA, cDNA, and mRNA sequences of viral, prokaryotic, eukaryotic and/or synthetic origin. The term also captures sequences that include any base analogs of DNA and RNA. The term “nucleic acid construct” as used herein, refers to a nucleic acid molecule containing the gene encoding the protein of interest and other 5′ or 3′ flanking regulatory sequences, such as a promoter, a leader sequence, an intron, an internal ribosome entry site (IRES) and a selectable marker gene.

A “nucleotide” refers to a subunit of DNA or RNA consisting of nitrogenous bases (adenine, guanine, cytosine and thymine), a phosphate molecule, and a sugar molecule (deoxyribose in DNA and ribose in RNA). The term is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U).

“Operably linked” refers to an arrangement of elements wherein the components so described are configured so as to perform their usual function. Thus, a given promoter that is operably linked to a coding sequence is capable of effecting the expression of the coding sequence when the regulatory proteins and proper enzymes are present. In some instances, certain control elements need not be contiguous with the coding sequence, so long as they function to direct the expression thereof.

The “polymerase chain reaction (PCR)” technique, is disclosed in U.S. Pat. Nos. 4,683,202, 4,683,195 and 4,800,159. In its simplest form, PCR is an in vitro method for the enzymatic synthesis of specific DNA sequences, using two oligonucleotide primers that hybridize to opposite strands and flank the region of interest in the target DNA. A repetitive series of reaction steps involving template denaturation, primer annealing and the extension of the annealed primers by DNA polymerase results in the exponential accumulation of a specific fragment (i.e, an amplicon) whose termini are defined by the 5′ ends of the primers. PCR is reported to be capable of producing a selective enrichment of a specific DNA sequence by a factor of 10⁹. The PCR method is also described in Saiki et al., 1985, Science, 230:1350. Other PCR methods, also applicable to the present invention, for example, RT-PCR, inverse PCR, quantitative PCR, real time PCR and in situ PCR are known to those skilled in the art.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. Moreover, an “oligonucleotide primer” refers to a single stranded DNA or RNA molecule that is hybridizable (eg. capable of annealing) to a nucleic acid template and is capable of priming enzymatic synthesis of a second nucleic acid strand. Alternatively, or in addition, oligonucleotide primers, when labeled directly or indirectly (e.g., bound by a labeled secondary probe which is specific for the oligonucleotide primer) may be used effectively as probes to detect the presence of a specific nucleic acid in a sample. Oligonucleotide primers useful according to the invention are between about 10 to about 100 nucleotides in length, about 17 to about 50 nucleotides in length, about 17 to about 40 nucleotides in length and more particularly about 17 to about 30 nucleotides in length.

The term “protein” as used herein refers to one or more polypeptides that can function as a unit. The term “polypeptide” as used herein refers to a sequential chain of amino acids linked together via peptide bonds. A therapeutic protein can be, for example, a secreted protein. Therapeutic proteins include antibodies, antigen-binding fragments of antibodies, soluble receptors, receptor fusions, cytokines, growth factors, enzymes, SMIPs, or clotting factors. The above list of proteins is merely exemplary in nature, and is not intended to be a limiting recitation. One of ordinary skill in the art will understand that any protein of interest may be expressed in accordance with the present invention and will be able to select the particular protein to be expressed as needed.

The term “rearrangement” as used herein, refers to any change in the introduced nucleic acid sequence, including those encoding, for example, a protein of interest. The rearrangement may occur by way of an addition or a deletion in one or more nucleotides in the gene encoding the protein of interest.

The term “recombinant” as used herein simply refers to any protein, or cell expressing a gene of interest that is produced by genetic engineering methods. In addition, “recombinant,” as used herein, further describes a nucleic acid molecule, which, by virtue of its origin or manipulation, is not associated with all or a portion of the polynucleotide with which it is associated in nature. The term “recombinant” as used with respect to a protein or polypeptide, means a polypeptide produced by expression of a recombinant polynucleotide. The term “recombinant” as used with respect to a host cell means a host cell into which a recombinant polynucleotide has been introduced.

In the present application, the term “selection marker” or “selectable marker” refers to a protein that facilitates the cloning and identification of transformants, for example, a protein that confer resistance to Apramycin, neomycin, puromycin, hygromycin, DHFR, GPT, zeocin, phleomycin, glutamine synthetase and histidinol are useful selectable markers. Accordingly, cells containing a nucleic acid construct of the present invention may be identified in vitro or in vivo by including in the construct a coding region for a selectable marker. Such markers would confer an identifiable change to the cell permitting easy identification of cells containing the nucleic acid construct. Generally, a selectable marker is one that confers a property that allows for selection. A positive selectable marker is one in which the presence of the marker allows for its selection, while a negative selectable marker is one in which its presence prevents its selection. An example of a positive selectable marker is a marker that confers drug resistance to a cell that expresses the marker. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (“tk”) or chloramphenicol acetyltransferase (“CAT”) may be utilized. One of skill in the art would also know how to employ immunologic markers, possibly in conjunction with FACS analysis. Any selectable marker may be used, so long as it is capable of being expressed simultaneously with the nucleic acid encoding a gene product. Further examples of selectable and screenable markers are well known to one of skill in the art.

“Small Modular Immunopharmaceutical” or (SMIP™) drugs (Trubion Pharmaceuticals, Seattle, Wash.). are single-chain polypeptides composed of a binding domain for a cognate structure such as an antigen, a counterreceptor or the like, a hinge-region polypeptide having either one or no cysteine residues, and immunoglobulin CH2 and CH3 domains (see also www.trubion.com). SMIPs and their uses and applications are disclosed in, e.g., U.S. Published Patent Application. Nos. 2007/002159, 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA. DNA-DNA or RNA-DNA. Such standard hybridization conditions are easily determined by one skilled in the art according to well known formulae, wherein hybridization is typically 10-20° C. below the predicted or determined Tm with washes of higher stringency, if desired. Defining appropriate hybridization conditions is within the skill of the art. See, e.g., Maniatis et al., supra; DNA Cloning, Vols. I & II, supra; Nucleic Acid Hybridization, supra.

### General Description

A vector used to drive expression of a gene in mammalian cells generally has several basic elements present. A promoter is required to drive gene expression and initiate transcription. An intronic sequence is required in the transcribed mRNA to enable efficient RNA processing and translation to occur. Finally, a polyadenylation sequence is needed to stabilize the RNA transcript.

An often used, and enabling, practice in the stable expression of foreign genes in mammalian cells is chemical selection. Chemical selection is a means for isolating cells that have taken up and integrated specific DNA. The DNA introduced encodes at least one protein, i.e., the selectable marker, whose function provides the ability to degrade or overcome a chemical selecting agent. For example, linking the DNA encoding a selectable marker to a gene of interest ensures that, so long as the cell can express the selectable marker to survive in the presence of the selecting agent, the cell will also continue to make the protein of interest (Kaufman, R J, et al. (1991), Nucleic Acids Res., Aug. 25; 19(16):4485-90).

Several approaches can be employed in linking the gene of interest to the DNA encoding a selectable marker: Co-introduction of DNA containing the gene of interest along with a second DNA containing the selectable marker (FIGS. 1A, B); introduction of a single piece of DNA containing the gene of interest and the selectable marker, expression of each being driven by its own respective promoter element (FIGS. 1A, B); or introduction of a single piece of DNA with the gene of interest and the selectable marker linked together in a single transcript with or without the use of an internal ribosomal entry site, or IRES, element (FIG. 1C). IRES' are typically used by viruses for efficient translation of the viral genome (Dobrikova, E Y, et al. (2006), J. Virology Apr; 80(7):3310-21). Incorporating an IRES into a gene expression cassette links the RNA for the protein of interest to the RNA for the selectable marker, thus providing a more direct mode of selection than having the RNA for the selectable marker and the RNA for the protein of interest as two separate transcripts.

Instability of gene expression is an event which hinders development of production cell lines for protein therapeutics. Loss of expression can occur through a variety of mechanisms including, but not limited to DNA methylation and chromosomal rearrangements including gene excision, homologous recombination and non-homologous recombination. Any of these mechanisms may lead to loss of the expression cassette, leaving the cell susceptible to the selecting agent.

One specific loss of expression is a rearrangement where by the DNA coding for the gene of interest is excised or “looped out.” This rearrangement may lead to an RNA transcript encoding only the selectable marker. The gene of interest is either completely removed or left in a non-functional state, i.e. incomplete transcript or non-sense sequence.

One object of the present invention is to identify a clonal population of cells containing a gene encoding a protein of interest that is suitable for large-scale production of the protein of interest. Studies were done to assess whether a high-throughput screen could be developed using a method such as polymerase chain reaction (PCR), to measure in a clonal population of cells the presence or absence of a rearranged gene encoding the protein of interest. If a rearrangement is observed in the gene of interest, this would indicate that the cells are not suitable for use in large-scale production of the protein of interest. The data demonstrated that such a gene rearrangement could be observed in cells expressing various proteins of interest, including an Fc-fusion protein, a SMIP and a monoclonal antibody. Moreover, when this genetic rearrangement is present in a clonal population of cells, this population of cells is shown to be unstable and thus unsuitable for large-scale production of the protein of interest.

### Uses of the Invention

As noted above, the invention provides methods for identifying a clonal population of cells suitable for large-scale production of a protein of interest. More particularly, the methods identify a clonal population of cells that have been transfected with a gene encoding the protein of interest, whereby the occurrence of a genetic rearrangement in the gene of interest indicates that the cells will not be suitable for scale-up production of the protein of interest encoded by that gene.

In one embodiment, the method of the invention comprises:
a) transfecting a population of cells with a nucleic acid construct comprising a gene encoding a protein of interest;
b) isolating a clonal population of cells expressing the gene encoding the protein of interest;
c) determining the presence or absence of a rearrangement of the gene encoding the protein of interest in the clonal population;
d) selecting a clonal population of cells from step c) that lack the rearrangement of the gene encoding the protein of interest; and
e) culturing the clonal population of cells from step d) for large-scale production of the protein of interest.

In another embodiment, the method comprises:
a) transfecting a population of cells with a nucleic acid comprising in sequential order a coding region for a tripartite leader sequence (TPL), an intron, a gene encoding a protein of interest, an IRES and a coding region for a selectable marker;
b) isolating a clonal population of cells expressing the gene encoding the protein of interest and the selectable marker;
c) determining the presence or absence of a rearrangement of the gene encoding the protein of interest in the clonal population;
d) selecting a clonal population of cells from step c) that lack the rearrangement of the gene encoding the protein of interest; and
e) culturing the clonal population of cells from step d) for large-scale production of the protein of interest.

The method further comprises isolating and purifying the protein of interest using any method known to those skilled in the art. The large-scale production comprises culturing the cells in a volume of cell culture medium that is at least two liters and may be 10, 100, 250, 500, 1,000, 2,500, 5,000, 8,000, 10,000, 12,000 liters or more, or any volume in between.

The methods of the invention may be used for identifying cells suitable for large-scale production of a protein of interest, whereby such protein may be any protein, such as a therapeutic protein, including an antibody or an antigen-binding fragment thereof. In some embodiments, the product is a secreted protein; a fusion protein, e.g., a receptor fusion protein or an Ig-fusion protein, including Fc-fusion proteins; a soluble receptor; a growth factor; an enzyme; a clotting factor; an Fc-containing protein; an immunoconjugate; a cytokine; an interleukin; a SMIP; a hormone; or a therapeutic enzyme.

Information on the aforementioned polypeptides, as well as many others, can be obtained from a variety of public sources, including electronic databases such as GenBank. A particularly useful site is the website of the National Center for Biotechnology Information/National Library of Medicine National Institutes of Health. Those of ordinary skill in the art are able to obtain information needed to express a desired polypeptide and apply the techniques described herein by routine experimentation.

### Host Cells

As used herein, the terms “cell”, or “cells”, or “host cells” may be used interchangeably. These terms also include their progeny, which is any and all subsequent generations. It is understood that all progeny may not be identical due to deliberate or inadvertent mutations. If the progeny are not genetically the same as the parent cell, then the cells are referred to as a “population of cells”, which is to be differentiated from a “clonal population of cells”. A “clonal population of cells”, as used herein, are different from the cells noted above in that the term generally refers to a population of cells that originated from a single isolated cell. As described In the context of expressing a heterologous nucleic acid sequence, “host cell” refers to a prokaryotic or eukaryotic cell, and more particularly in the present invention, to a eukaryotic cell, and it includes any transformable organism that is capable of replicating a vector and/or expressing a heterologous gene encoded by a vector. A host cell can, and has been, used as a recipient for vectors. A host cell may be “transfected” or “transformed,”, which refers to a process by which exogenous nucleic acid is transferred or introduced into the host cell. A transformed cell includes the primary subject cell and its progeny. Some expression vectors of the present invention may employ control sequences that allow it to be replicated and/or expressed in both prokaryotic and eukaryotic cells. One of skill in the art would further understand conditions under which to incubate such host cells to maintain them and to permit replication of a vector. Also understood and known are techniques and conditions that would allow large-scale production of vectors, as well as production of the nucleic acids encoded by vectors and their cognate polypeptides, proteins, or peptides.

Transfected host cells are cells which have been transfected (sometimes referred to as transformed) with heterologous DNA. Many techniques for transfecting cells are known; in one approach, cells are transfected with expression vectors constructed using recombinant DNA techniques and which contain sequences encoding recombinant proteins. Expressed proteins will preferably be secreted into the culture supernatant, but may be associated with the cell membrane, depending on the particular polypeptide that is expressed. Mammalian host cells are preferred for the instant invention. Various mammalian cell culture systems can be employed to express recombinant protein or can be mammalian production cells adapted to grow in cell culture, and/or can be homogenous cell lines. Examples of such cells commonly used in the industry are VERO, BHK, HeLa, CV1 (including Cos), MDCK, 293, 3T3, myeloma cell lines (e.g., NSO, NS1), PC12, WI38 cells, and Chinese hamster ovary (CHO) cells, which are widely used for the production of several complex recombinant polypeptides, e.g. cytokines, clotting factors, and antibodies (Brasel et al. (1996), Blood 88:2004-2012; Kaufman et al. (1988), J. Biol Chem 263:6352-6362; McKinnon et al. (1991), J Mol Endocrinol 6:231-239; Wood et al. (1990), J. Immunol. 145:3011-3016). The dihydrofolate reductase (DHFR)-deficient mutant cell lines (Urlaub et al. (1980), Proc Natl Acad Sci USA 77: 4216-4220, which is incorporated by reference), DXB11 and DG-44, are desirable CHO host cell lines because the efficient DHFR selectable and amplifiable gene expression system allows high level recombinant polypeptide expression in these cells (Kaufman R. J. (1990), Meth Enzymol 185:537-566, which is incorporated by reference). In addition, these cells are easy to manipulate as adherent or suspension cultures and exhibit relatively good genetic stability.

A commonly used cell line is DHFR-CHO cells which are auxotrophic for glycine, thymidine and hypoxanthine, and can be transformed to the DHFR-t phenotype using DHFR cDNA as an amplifiable dominant marker. One such DHFR-CHO cell line, DXB11, was described by Urlaub and Chasin (Proc. Natl. Acad. Sci. 30 USA 77:4216, 1980). Another example of a DBFR-CHO cell line is DG44 (see, for example, Kaufman, R. J., Meth. Enzymology 185:537 (1988)). Other cell lines developed for specific selection or amplification schemes will also be useful with the invention. CHO cells and recombinant polypeptides expressed in them have been extensively characterized and have been approved for use in clinical commercial manufacturing by regulatory agencies. The methods of the invention can also be practiced using hybridoma cell lines that produce an antibody. Methods for making hybridoma lines are well known in the art. See e.g. Berzofsky et al. in Paul, ed., Fundamental Immunology, Second Edition, pp. 315-356, at 347-350, Raven Press Ltd., New York (1989). Cell lines derived from the above-mentioned lines are also suitable for practicing the invention.

Numerous other eukaryotic cells will also be useful in the present invention, including cells from other vertebrates, and insect cells. Those of skill in the art will be able to select appropriate vectors, regulatory elements, transfection and culture schemes according to the needs of their preferred culture system.

### Preparation of Transfected Mammalian Cells

Several transfection protocols are known in the art, and are reviewed in Kaufman, R. J. The transfection protocol chosen will depend on the host cell type and the nature of the protein of interest, and can be chosen based upon routine experimentation. The basic requirements of any such protocol are first to introduce a gene encoding the protein of interest, e.g. a heterologous DNA into a suitable host cell, and then to identify and isolate host cells which have incorporated the heterologous DNA in a stable, expressible manner.

One commonly used method of introducing heterologous DNA is calcium phosphate precipitation, for example, as described by Wigler et al. (Proc. Natl. Acad. Sci. USA 77:3567, 1980).

Polyethylene-induced fusion of bacterial protoplasts with mammalian cells (Schaffner et al., Proc. Natl. Acad. Sci. USA 77:2163, 1980) is another useful method of introducing heterologous DNA. Protoplast fusion protocols frequently yield multiple copies of the plasmid DNA integrated into the mammalian host cell genome. This technique requires the selection and amplification marker to be on the same plasmid as the gene of interest.

Electroporation can also be used to introduce DNA directly into the cytoplasm of a host cell, as described by Potter et al. (Proc. Natl. Acad. Sci. USA 81:7161, 1988) or Shigeltawa and Dower (BioTechniques 6:742, 1988). Unlike protoplast fusion, electroporation does not require the selection marker and the gene of interest to be on the same plasmid.

More recently, several reagents useful for introducing heterologous DNA into a mammalian cell have been described. These include Lipofectin™ Reagent and Lipofectamine™ Reagent (Gibco BRL, Gaithersburg, Md.). Both of these reagents are commercially available reagents used to form lipid-nucleic acid complexes (or liposomes) which, when applied to cultured cells, facilitate uptake of the nucleic acid into the cells.

Transfection of cells with heterologous DNA and selection for cells that have taken up the heterologous DNA and express the selectable marker results in a pool of transfected cells. Individual cells in these pools will vary in the amount of DNA incorporated and in the chromosomal location of the transfected DNA. After repeated passage, pools frequently lose the ability to express the heterologous protein. To generate a stable clonal population of cells, individual cells can be isolated from the pools and cultured (a process referred to as cloning), a laborious time consuming process. However, in some instances, the pools them selves may be stable (ie., production of the heterologous recombinant protein remains stable).

Even a stable clonal population of cells, however, may lose the ability to express the heterologous protein over time. This loss of expression can be the result of a rearrangement in the gene encoding the protein of interest. Without wishing to be bound by a particular theory, it is believe that a subset of cells in the population that undergo this rearrangement acquire a selective advantage over the non-rearranged cells in the population, and overtime will thereby predominate in the clonal population. The ability to select and culture non-rearranged stable pools of cells would be desirable as it would allow for continuous, large-scale production of a protein of interest from a clonal population of cells.

A method of amplifying the gene of interest is also desirable for expression of the recombinant protein, and typically involves the use of a selection marker. Resistance to cytotoxic drugs is the characteristic most frequently used as a selection marker, and can be the result of either a dominant trait (i.e., can be used independent of host cell type) or a recessive trait (i.e., useful in particular host cell types that are deficient in whatever activity is being selected for). Several amplifiable markers are suitable for use in the inventive expression vectors (for example, as described in Maniatis, Molecular Biology: A Laboratory Manual, Cold Spring Harbor Laboratory, NY, 1989; pgs 16.9-16.14).

Useful selectable markers for gene amplification in drug-resistant mammalian cells include dihydrofolate reductase-methotrexate (DHFR-MTX) resistance, P-glycoprotein and multiple drug resistance (MDR)-various lipophilic cytoxic agents (i.e., adriamycin, colchicine, vincristine), and adenosine deaininase (ADA)-Xyl-A or adenosine and 2′-deoxycoformycin. Specific examples of genes that encode selectable markers are those that encode antimetabolite resistance such as the DHFR protein, which confers resistance to methotrexate (Wigler et al., 1980, Proc. Natl. Acad. Sci. USA 77:3567; O'Hare et al., 1981, Proc. Natl. Acad. Sci. USA 78:1527); the GPT protein, which confers resistance to mycophenolic acid (Mulligan & Berg, 198 1, Proc. Natl. Acad. Sci. USA 78:2072), the neomycin resistance marker, which confers resistance to the aminoglycoside G-418 (Colberre-Garapin et al., 1981, J. Mol. Biol. 150: 1); the Hygro protein, which confers resistance to hygromycin 5 (Santerre et al., 1984. Gene 30:147); and the zeocin™ resistance marker (available commercially from Invitrogen). In addition, the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22: 8 17) genes can be employed in tk-, hgprt- and aprt-cells, respectively.

Other dominant selectable markers include microbially derived antibiotic resistance genes, for example neomycin, kanamycin or hygromycin resistance. However, these selection markers have not been shown to be amplifiable (Kaufman, R. J., supra,). Several suitable selection systems exist for mammalian hosts (Maniatis supra, pgs 16.9-16.15). Co-transfection protocols employing two dominant selectable markers have also been described (Okayama and Berg, Mol Cell Biol 5:1136, 1985). A particularly useful selection and amplification scheme utilizes dihydrofolate reductase (DHFR) methotrexate (MTX) resistance (DHFR-MTX). MTX is an inhibitor of DHFR that has been shown to cause amplification of endogenous DHFR genes (Alt F. W., et al., J Biol Chem 253:1357, 1978) and transfected DHFR sequences (Wigler M., et al., Proc. Natl. Acad. Sci. USA 77:3567, 1980). In one embodiment, cells are transfected with DNA comprising the gene of interest and DNA encoding DHFR in a bicistronic expression unit (Kauhan et al., 1991 supra and Kaufman R. J., et al., EMBO J 6: 187, 1987). Transfected cells are grown in media containing successively greater amounts of MTX, resulting in greater expression of the DHFR gene, as well as the gene of interest.

Useful regulatory elements, described previously, can also be included in the plasmids or expression vectors used to transfect mammalian cells. The transfection protocol chosen, and the elements selected for use therein, will depend on the type of host cell used. Those of skill in the art are aware of numerous different protocols and host cells, and can select an appropriate system for expression of a desired protein, based on the requirements of their selected cell culture system(s).

### Regulatory Elements

As used herein, regulatory elements and/or regulatory sequences are nucleotide sequences that enhance or otherwise modulate transcription and/or translation or that stabilize transcription and/or translation products. Thus, for example, promoters operably linked to a coding sequence of an expression construct enhance transcription of that coding sequence.

Exemplary regulatory elements can include, without limitation, promoters, enhancers, introns, termination sequences, polyadenylation sequences, stabilization sequences and the like. Some suitable regulatory sequences useful in the present invention will include, but are not limited to constitutive promoters, tissue-specific promoters, development-specific promoters, inducible promoters and viral promoters.

In certain embodiments, the nucleic acid encoding a protein of interest is operably linked and under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

In certain embodiments of the invention, the human cytomegalovirus (CMV) promoter, the SV40 early promoter, the Rous sarcoma virus (RSV) long terminal repeat, rat insulin promoter or glyceraldehyde-3-phosphate dehydrogenase promoter can be used to obtain high-level expression of the coding sequence of interest. The use of other viral or mammalian cellular or bacterial phage promoters which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. By way of illustration, a ubiquitous, strong (i.e., high activity) promoter may be employed to provide abundant gene expression in a group of host cells, or a tissue-specific promoter may be employed to target gene expression to one or more specific cell types. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole is typically able to stimulate transcription at a distance; this need not be true of a promoter region or its component elements. On the other hand, a promoter typically has one or more elements that direct initiation of RNA synthesis at a particular site and in a particular orientation, whereas enhancers generally lack these specificities. Promoters and enhancers are often overlapping and contiguous, often seeming to have a very similar modular organization.

Other promoter/enhancer combinations (see, e.g., the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct. In one aspect, tissue-specific promoters, e.g., cardiac-specific and/or fibroblast-specific promoters, are of particular interest. By way of illustration, cardiac-specific promoters include the myosin light chain-2 promoter (Franz et al., 1994, Circ Res. 1993 October; 73(4):629-38; Kelly et al., 1995, J Cell Biol. 1995 April; 129(2):383-96), the alpha actin promoter (Moss et al., 1996, J Biol Chem. 1996 Dec. 6; 271(49):31688-94), the troponin 1 promoter (Bhavsar et al, 1996, Genomics. 1996 Jul. 1; 35(1):11-23), the dystrophin promoter (Kimura et al., 1997, Dev Growth Differ. 1997 June; 39(3):257-65), the creatine kinase promoter (Ritchie, M. E., 1996, J Biol Chem. 1996 Oct. 11; 271(41):25485-91), the alpha7 integrin promoter (Ziober & Kramer, 1996, J Biol Chem. 1996 Sep. 13; 271(37):22915-22), the brain natriuretic peptide promoter (LaPointe et al, 1996, Hypertension. 1996 March; 27(3 Pt 2):715-22) and the alpha B-crystallin/small heat shock protein promoter (Gopal-Srivastava, R., 1995, Mol Cell Biol. 1995 December; 15(12):7081-90), alpha myosin heavy chain promoter (Yamauchi-Takihara et al., 1989, PNAS, May 15, 1989, vol. 86 (10): 3504-3508) and the ANF promoter (LaPointe et al., 1996, Hypertension, 27: 715-722).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read though from the cassette into other sequences.

### Selectable Markers

In certain embodiments of the invention, in which cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to ampicillin, neomycin, puromycin, hygromycin, DWR, GPT, zeocin and histidinol are useful selectable markers, as are those noted above. Alternatively, enzymes such as herpes simplex virus thymidine kinase (tk) or chloramphenicol acetyltransferase (CAT) may be employed. Immunologic markers also can be employed. The selectable marker should be capable of being expressed simultaneously with the nucleic acid encoding a gene product. Further examples of selectable markers are well known to one of skill in the art.

### Methods for Delivering a Nucleic Acid to a Cell

A “vector” is a DNA molecule, capable of replication in a host organism, into which a nucleic acid sequence is inserted to construct a recombinant DNA molecule. The nucleic acid sequence can be “exogenous” (e.g. foreign to the cell into which it is introduced), or “endogenous” (e.g., the same as a sequence in the cell into which it is introduced). Exemplary vectors include plasmids, cosmids, viruses (bacteriophage, animal viruses, and plant viruses), and artificial chromosomes (e.g., YACs), lipid-based vectors (e.g., liposomes) and other macromolecular complexes capable of mediating delivery of a polynucleotide to a host cell. (Schek, N, Cooke, C., and J. C. Alwine (1992), Mol. Cell Biol. 12:5386-5393; Klasens, B. I. F., Das, A. T., and B. Berkhout (1998): Nucleic Acids Res. 26: 1870-1876; Gil, A., and N. J. Proudfoot. (1987): Cell 49:399-406; Cole, C. N. and T. P. Stacy (1985): Mol. Cell. Biol. 5:2104-2113; Batt, D. B and G. G. Carmichael (1995): Mol. Cell. Biol. 15:4783-4790; Gimmi, E. R., Reff, M. E., and I. C. Deckman. (1989): Nucleic Acids Res. 17:6983-6998). One of skill in the art would be well equipped to construct a vector through standard techniques, for example standard recombinant techniques such as described in Sambrook et al., 1989 and Ausubel et al., 1994, both incorporated herein by reference.

A large number of viral and non-viral vectors (including lipid-based and other synthetic delivery systems known in the art) can likewise be employed to deliver polynucleotides of the present invention. Such vectors may be modified, as known to those of skill in the art, to confer or enhance cell specificity. By way of illustration, the surface of viral vectors may be modified such that they preferentially or exclusively bind to and/or infect a particular target cell population.

As described herein, an expression vector of the invention includes a vector containing a nucleic acid sequence coding for at least part of a gene product capable of being transcribed. In some cases, the transcription product(s) are then translated into a protein, polypeptide, or peptide. In other cases, these sequences are not translated, for example, in the production of antisense molecules or ribozymes. Expression vectors can contain a variety of “regulatory elements and/or control sequences,” which refer to nucleic acid sequences that regulate the transcription and possibly translation of an operably linked coding sequence in a particular host organism. In addition to control sequences that govern transcription and translation, vectors and expression vectors may contain nucleic acid sequences that serve other functions as well for example as described herein.

Recombinant expression vectors may include a coding sequence encoding a protein of interest, e.g., a therapeutic protein, (or fragment thereof), ribozymes, ribosomal mRNAs, antisense RNAs and the like. The coding sequence may be synthetic, a cDNA-derived nucleic acid fragment or a nucleic acid fragment isolated by polymerase chain reaction (PCR).

Expression vectors may also comprise non-transcribed elements such as a suitable promoter and/or enhancer linked to the gene to be expressed, other 5′ or 3′ flanking non-transcribed sequences, 5′ or 3′ non-translated sequences such as ribosome binding sites, a polyadenylation site, splice donor and acceptor sites, and transcriptional termination sequences. An origin of replication that confers the ability to replicate in a host, and a selectable gene to facilitate recognition of transfectants, may also be incorporated.

DNA regions are operably linked when they are functionally related to each other. For example, DNA for a signal peptide (secretory leader) is operably linked to DNA for a polypeptide if it is expressed as a precursor which participates in the secretion of the polypeptide; thus, in the case of DNA encoding secretory leaders, operably linked means contiguous and in reading frame. A promoter is operably linked to a coding sequence if it controls the transcription of the sequence; and a ribosome binding site is operably linked to a coding sequence if it is positioned so as to permit translation.

The transcriptional and translational control sequences in expression vectors to be used in transfecting cells may be provided by viral sources. For example, commonly used promoters and enhancers are derived from Polyoma, Adenovirus 2, Simian Virus 40 (SV40), and human cytomegalovirus. Viral genomic promoters, control and/or signal sequences may be utilized to drive expression, provided such control sequences are compatible with the host cell chosen. Examples of such vectors can be constructed as disclosed by Okayama and Berg (Mol. Cell. Biol. 3:280, 1983). Non-viral cellular promoters can also be used (i.e., the beta-globin and the EF-Ia promoters), depending on the cell type in which the recombinant protein is to be expressed.

DNA sequences derived from the SV40 viral genome, for example, SV40 origin, early and late promoter, enhancer, splice, and polyadenylation sites may be used to provide the other genetic elements required for expression of a heterologous DNA sequence. The early and late promoters are particularly useful because both are obtained easily from the virus as a fragment which also contains the SV40 viral origin of replication (Fiers et al., Nature 273: 1 13, 1978). Smaller or larger SV40 fragments may also be used.

An additional technique that can be used in conjunction with an expression vector is described by Lucas et al. (Nucleic Acids Res. 24: 1774; 1996). In an effort to increase production of a desired protein, Lucas et al. utilized mRNA splice donor and acceptor sites to develop stable clones that produced both a selectable marker and recombinant proteins. According to these investigators, the vectors they prepared resulted in the transcription of a high proportion of mRNA encoding the desired protein, and a fixed, relatively low level of the selection marker that allowed selection of stable transfectants.

### Expression Systems

Mammalian cells suitable for carrying out the present invention include, but are not limited to, VERO, BHK, HeLa, CV1 (including Cos), MDCK, 293, 3T3, myeloma cell lines (e.g., NSO, NS1), PC12, WI38 cells, and Chinese hamster ovary (CHO) cells. As noted above, suitable expression vectors for directing expression in mammalian cells generally include a promoter, as well as other transcriptional and translational regulatory and/or control sequences. Representative methods include calcium phosphate mediated gene transfer, electroporation, retroviral, and protoplast fusion mediated transfection (see, for example, Sambrook et al.). Numerous expression systems exist that comprise at least a part or all of the compositions described herein. Various eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be purchased, for example, under the name MAXBACO 2.0 from INVITROGEN and BACPACK™ baculovirus expression system from CLONTECH.

Other examples of expression systems include STRATAGENE'S COMPLETE CONTROL™ Inducible Mammalian Expression System, or its PET Expression System, an E. coli expression system. Another example of an inducible expression system is available from INVITROGEN, which carries the T-REX™ (tetracycline-regulated expression) System, an inducible mammalian expression system that uses the full-length CMV promoter. INVITROGEN also provides a yeast expression system, which is designed for high-level production of recombinant proteins in the methylotrophic yeast Pichia methanolica.

One of skill in the art would know how to express a vector, such as an expression construct, to produce a nucleic acid sequence or its cognate polypeptide, protein, or peptide.

### IRES' Useful in the Invention

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988, Nature, 334:320-325). IRES elements from two members of the picornavirus family (polio and encephalomyocarditis) have been described (Pelletier and Sonenberg, 1988, Nature, 334:320-325), as well an IRES from a mammalian message (Macejak and Sarnow, 1991; Nature, Sep. 5; 353(6339):90-4.). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference). It is envisioned that any IRES element may be used in the methods of the present invention. For example, particular IRES' are shown in SEQ ID NOs: 4, 6 and 8. Other IRES' may be purchased from, for example, CLONTECH (See catalogue numbers 631605, 631607, 631619, 631620, 631621 and 631622).

An Internal Ribosome Entry Site (IRES) is described in: Morgan R A, Couture L, Elroy-Stein O, Ragheb J, Moss B, Anderson W F. Retroviral vectors containing putative internal ribosome entry sites: development of a polycistronic gene transfer system and applications to human gene therapy. Nucleic Acids Res. 1992, Mar. 25; 20(6):1293-9. An example of an IRES is shown in EMC Genbank accession number: AJ000155 starting from base 767 through base 1310. Other IRES sequences are found in Genbank accession numbers V01149, from base number 1 to 627 and also in Genbank accession number NC_{—}001461 from base 1 to 900.

### Primers Useful for Practicing the Methods of the Invention

The invention provides a method for identifying a clonal population of cells suitable for large-scale production of a protein of interest. More particularly, the method provides for transfecting a population of cells with a nucleic acid construct comprising a gene encoding a protein of interest, isolating a clonal population of cells encoding the protein of interest and determining the presence or absence of a rearrangement of the gene encoding the protein of interest prior to large scale production of the protein. In one embodiment, the rearrangement is determined by detecting a deletion of all or part of the gene encoding the protein of interest. In one embodiment, the deletion is detected by amplification of a nucleic acid region that, in the absence of a rearrangement, includes the gene encoding the protein of interest as well as nucleic acid regions 5′ and 3′ of said gene. In accordance with this procedure, it is contemplated, and demonstrated herein, that particular primers that bind to a region 5′ and 3′ to the gene of interest are suitable for use. In one embodiment, the primers may bind to a site within the leader sequence, such as the tripartite leader sequence used herein (which, in the present invention, is 5′ to the gene encoding the protein of interest) and to a site within the marker gene (which, in the present invention, is 3′ to the gene encoding the protein of interest). In the present invention, the primers shown as SEQ ID NOs: 1 and 2 were utilized for this purpose. However, any primer may be designed that binds to any other region 5′ and 3′ to the gene encoding the protein of interest. Determining the presence or absence of the full length gene or the rearranged gene may be done using any amplification procedure known to those skilled in the art, such as, but not limited to, a polymerase chain reaction (PCR). The size of the gene encoding the protein of interest or the rearranged gene (the deletion of all or part of the gene encoding the protein of interest) may then be monitored using, for example, agarose gel analysis. Accordingly, the size of the gene encoding the protein of interest determines whether a rearrangement has occurred in a clonal population of cells. In accordance with the invention, a clonal population of cells suitable for large-scale production of a protein of interest is selected if a rearrangement of the gene encoding the protein of interest is absent.

Oligonucleotide primers useful according to the invention may be single-stranded DNA or RNA molecules that are hybridizable to a template nucleic acid sequence and prime enzymatic synthesis of a second nucleic acid strand. The primer is complementary to a portion of a target molecule present in a pool of nucleic acid molecules. It is contemplated that oligonucleotide primers according to the invention may be prepared by synthetic methods, either chemical or enzymatic. Alternatively, such a molecule or a fragment thereof may be naturally-occurring, and is isolated from its natural source or purchased from a commercial supplier. Oligonucleotide primers are generally 5 to 100 nucleotides in length, ideally from 17 to 40 nucleotides, although primers of different lengths may also be used. Primers for amplification are preferably about 17-25 nucleotides. Primers useful according to the invention are also designed to have a particular melting temperature (Tm) by the method of melting temperature estimation. Commercial programs, including Oligo™, Primer Design and programs available on the internet, including Primer3 and Oligo Calculator can be used to calculate a Tm of a nucleic acid sequence useful according to the invention. Preferably, the Tm of an amplification primer useful according to the invention, as calculated for example by Oligo Calculator, is preferably between about 45 and 65° C. and more preferably between about 50° and 60° C.

Typically, selective hybridization occurs when two nucleic acid sequences are substantially complementary (at least about 65% complementary over a stretch of at least 14 to 25 nucleotides, preferably at least about 75%, more preferably at least about 90% complementary). See Kanehisa, M., 1984, Nucleic Acids Res. 12: 203, incorporated herein by reference. As a result, it is expected that a certain degree of mismatch at the priming site is tolerated. Such mismatch may be small, such as a mono-, di- or tri-nucleotide. Alternatively, a region of mismatch may encompass loops, which are defined as regions in which there exists a mismatch in an uninterrupted series of four or more nucleotides.

Numerous factors influence the efficiency and selectivity of hybridization of the primer to a second nucleic acid molecule. These factors, which include primer length, nucleotide sequence and/or composition, hybridization temperature, buffer composition and potential for steric hindrance in the region to which the primer is required to hybridize, will be considered when designing oligonucleotide primers according to the invention.

A positive correlation exists between primer length and both the efficiency and accuracy with which a primer will anneal to a target sequence. In particular, longer sequences have a higher melting temperature (TM) than do shorter ones, and are less likely to be repeated within a given target sequence, thereby minimizing promiscuous hybridization. Primer sequences with a high G-C content or that comprise palindromic sequences tend to self-hybridize, as do their intended target sites, since unimolecular, rather than bimolecular, hybridization kinetics are generally favored in solution. However, it is also important to design a primer that contains sufficient numbers of G-C nucleotide pairings since each G-C pair is bound by three hydrogen bonds, rather than the two that are found when A and T bases pair to bind the target sequence, and therefore forms a tighter, stronger bond. Hybridization temperature varies inversely with primer annealing efficiency, as does the concentration of organic solvents, e.g. formamide, that might be included in a priming reaction or hybridization mixture, while increases in salt concentration facilitate binding. Under stringent annealing conditions, longer hybridization probes, or synthesis primers, hybridize more efficiently than do shorter ones, which are sufficient under more permissive conditions. Stringent hybridization conditions typically include salt concentrations of less than about 1M, more usually less than about 500 mM and preferably less than about 200 mM. Hybridization temperatures range from as low as 0° C. to greater than 22° C. greater than about 30° C., and (most often) in excess of about 37° C. Longer fragments may require higher hybridization temperatures for specific hybridization. As several factors affect the stringency of hybridization, the combination of parameters is more important than the absolute measure of a single factor.

Oligonucleotide primers can be designed with these considerations in mind and synthesized according to the following methods.

### Oligonucleotide Primer Design Strategy

The design of a primer is facilitated by the use of readily available computer programs, developed to assist in the evaluation of the several parameters described above and the optimization of primer sequences. Examples of such programs are “Primer Express” (Applied Biosystems), “PrimerSelect” of the DNAStar™. “PrimerSelect” of the DNAStar.™. software package (DNAStar, Inc.; Madison, Wis.), OLIGO 4.0 (National Biosciences, Inc.), PRIMER, Oligonucleotide Selection Program, PGEN and Amplify (described in Ausubel et al., 1995, Short Protocols in Molecular Biology, 3rd Edition, John Wiley & Sons).

### Large-Scale Production of the Protein of Interest

According to the present invention, a mammalian host cell is cultured under conditions that promote the production of the protein of interest, e.g., an antibody, an Fc fusion protein or a SMIP. Basal cell culture medium formulations are well known in the art. To these basal culture medium formulations the skilled artisan will add components such as amino acids, salts, sugars, vitamins, hormones, growth factors, buffers, antibiotics, lipids, trace elements and the like, depending on the requirements of the host cells to be cultured. The culture medium may or may not contain serum and/or protein. Various tissue culture media, including serum-free and/or defined culture media, are commercially available for cell culture. Tissue culture media is defined, for purposes of the invention, as a media suitable for growth of animal cells, and preferably mammalian cells, in in vitro cell culture. Typically, tissue culture media contains a buffer, salts, energy source, amino acids, vitamins and trace essential elements. Any media capable of supporting growth of the appropriate eukaryotic cell in culture can be used; the invention is broadly applicable to eukaryotic cells in culture, particularly mammalian cells, and the choice of media is not crucial to the invention. Tissue culture media suitable for use in the invention are commercially available from, e.g., ATCC (Manassas, Va.). For example, any one or combination of the following media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL.™. 300 Series (available from JRH Biosciences, Lenexa, Kans., USA), among others, which can be obtained from the American Type Culture Collection or JRH Biosciences, as well as other vendors. When defined medium that is serum-free and/or peptone-free is used, the medium is usually highly enriched for amino acids and trace elements. See, for example, U.S. Pat. No. 5,122,469 to Mather et al. and U.S. Pat. No. 5,633,162 to Keen et al.

Suitable culture conditions for mammalian cells are known in the art. See e.g. Animal cell culture: A Practical Approach, D. Rickwood, ed., Oxford university press, New York (1992). Mammalian cells may be cultured in suspension or while attached to a solid substrate. Furthermore, mammalian cells may be cultured, for example, in fluidized bed bioreactors, hollow fiber bioreactors, roller bottles, shake flasks, or stirred tank bioreactors, with or without microcarriers, and operated in a batch, fed batch, continuous, semi-continuous, or perfusion mode.

The only process, which is economically viable is a reactor process because the scale-up can be made appropriate to the market size and the amount of the product needed. For adherent cells the carrier process with a classical microcarrier is currently the best choice for large scale cultivation of the cells needed for protein production (Van Wezel et al. 1967. Nature 216:64-65; Van Wezel et al. 1978. Process Biochem. 3:6-8).

According to one embodiment, the method provides for use of CHO cells, although any other cell suitable for protein production on a large scale, as known to those skilled in the art, may be used. See, for example, U.S. Pat. Nos. 6,872,549; 6,855,535 and 6,951,752, all incorporated by reference in their entireties.

Adherent cells bound to a microcarrier can be grown in conventional culture medium containing serum. In one embodiment of the invention, the cells are grown in serum free or serum and protein free medium as described by Kistner et al. (1998. Vaccine 16: 960-968), Merten et al. (1994. Cytotech. 14:47-59), Cinatl. et al. (1993. Cell Biology Internat. 17:885-895), Kessler et al. (1999. Dev. Biol. Stand. 98:13-21), WO 96/15231, U.S. Pat. No. 6,100,061 or any other serum free or serum and protein free medium known in the art. The cells are preferably grown from an ampoule to a large scale to a biomass in serum free or serum and protein free medium.

A microcarrier that may be used according to the method of the invention may be selected from the group of microcarriers based on dextran, collagen, polystyrene, polyacrylamide, gelatine, glass, cellulose, polyethylene and plastic and those described by Miller et al. (1989. Advances in Biochem Eng./Biotech. 39:73-95) and described in Butler (1988. In: Spier & Griffiths, Animal cell Biotechnology 3:283-303).

It is within the knowledge of one skilled in the art to select the respective microcarrier type, the microcarrier concentration in the starting culture, the adherent cells susceptible to the virus or vector used, and the medium and optimal growth conditions, like oxygen concentration, supplements of the medium, temperature, pH, pressure, steering speed and feeding control, to obtain a confluent cell culture biomass which can be used to obtain a cell biomass having increased cell density and microcarrier concentration according to this method. The cell culture having higher cell density biomass can then be used then for effective protein production. After the cell culture has reached confluency, the method of the invention allows to obtain a cell culture having an increased cell density of microcarrier concentration of at least 1,3-fold up to 10 fold and obtain higher protein yield per culture volume due i) reduced culture volume and ii) increased productivity per cell.

### Isolation and Purification of the Protein of Interest

The resulting expressed polypeptide can be collected, isolated and purified, or partially purified, from such culture or component (e.g., from culture medium or cell extracts) using known processes. By “partially purified” means that some fractionation procedure, or procedures, have been carried out, but that more polypeptide species (at least 10%) than the desired polypeptide is present. By “purified” is meant that the polypeptide is essentially homogeneous, i.e., less than 1% contaminating polypeptides are present. Fractionation procedures can include but are not limited to one or more steps of filtration, centrifugation, precipitation, phase separation, affinity purification, gel filtration, ion exchange chromatography, hydrophobic interaction chromatography (HIC; using such resins, as phenyl ether, butyl ether, or propyl ether). HPLC, or some combination of above.

For example, the purification of the polypeptide can include an affinity column containing agents which will bind to the polypeptide; one or more column steps over such affinity resins as concanavalin A-agarose, heparin-TOYOPEARL.®. (Toyo Soda Manufacturing Co. Ltd., Japan) or Cibacrom blue 3GA SEPHAROSE.®. (Pharmacia Fine Chemicals, Inc., New York); one or more steps involving elution; and/or immunoaffinity chromatography. The polypeptide can be expressed in a form that facilitates purification. For example, it may be expressed as a fusion polypeptide, such as those of maltose binding polypeptide (MBP), glutathione-S-transferase (GST), or thioredoxin (TRX). Kits for expression and purification of such fusion polypeptides are commercially available from New England BioLab (Beverly, Mass.), Pharmacia (Piscataway, N.J.) and InVitrogen, respectively. The polypeptide can be tagged with an epitope and subsequently purified by using a specific antibody directed to such epitope. One such epitope (FLAGS is commercially available from Kodak (New Haven, Conn.). It is also possible to utilize an affinity column comprising a polypeptide-binding protein, such as a monoclonal antibody to the recombinant polypeptide, to affinity-purify expressed polypeptides. Other types of affinity purification steps can be a Protein A or a Protein G column, which affinity agents bind to proteins that contain Fc domains. Polypeptides can be removed from an affinity column using conventional techniques, e.g., in a high salt elution buffer and then dialyzed into a lower salt buffer for use or by changing pH or other components depending on the affinity matrix utilized, or can be competitively removed using the naturally occurring substrate of the affinity moiety.

The desired degree of final purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Optionally, the polypeptide of the invention is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography. In certain embodiments, the purified polypeptide is formulated for therapeutic use.

### EXAMPLES

The following examples demonstrate certain aspects of the present invention. However, it is to be understood that these examples are for illustration only and do not purport to be wholly definitive as to conditions and scope of this invention. It should be appreciated that when typical reaction conditions (e.g., temperature, reaction times, etc.) have been given, the conditions both above and below the specified ranges can also be used, though generally less conveniently. The examples are conducted at room temperature (about 23° C. to about 28° C.) and at atmospheric pressure. All parts and percents referred to herein are on a weight basis and all temperatures are expressed in degrees centigrade unless otherwise specified.

### Materials and Methods

The following Materials and Methods were employed for Examples 1 through 3.

### Cell Culture

CHO cells were transfected with a gene encoding a protein of interest within the vector context outlined in FIG. 1C. Individual clones are isolated using methotrexate as a selecting agent. Clones were expanded in 96-well plates and screened for protein expression. A subset of high expressing clones from this screen was then moved into a chemically-defined medium in suspension. Cells were screened again (secondary screen) for protein expression and a further subset was expanded and monitored for protein expression every passage.

### RNA Isolation

During the secondary screen, RNA was isolated from 1×10⁶-5×10⁶ cells from each individual clone using the Qiagen RNeasy kit according to the manufacturer's instructions (Qiagen Inc, USA).

### Reverse Transcription Polymerase Chain Reaction

RNA from individual clones was used as a template for RT-PCR using the AccessQuick RT-PCR kit (Promega, Madison Wis.) according to the manufacturer's instructions. This is a single step RT-PCR reaction kit, which means that a 2×reaction buffer contains materials needed for the PCR reaction. Briefly, for each clone, 1 μg of RNA was used per reaction. Each reaction tube contained 50 pmol of the forward oligo “Int Gen F” (5′-TACTCTTGGATCGGAAACCCGTCG-3′ (SEQ ID NO: 1)), which anneals to the tripartite leader sequence in the expression cassette. Each reaction tube contained 50 pmol of the reverse oligo “DHFR2” (5′-CTACTTTACTTGCCAATTCC-3′ (SEQ ID NO: 2)), which anneals to the mouse DHFR sequence in the expression cassette. These sequences flank the gene of interest. Reverse transcription used 1-2 units of the AMV reverse transcriptase, provided by the manufacturer. The total reaction volume was 50 μl. The reaction conditions are as follows: 1 cycle at 45° C. for 1.5 h; 1 cycle at 95° C. for 5 min; 28 cycles of 94° C. for 1 min, 46° C. for 1 min, 68° C. for 3 min; and finally 1 cycle at 68° C. for 7 min. The completed PCR product was separated on 1-2% agarose gels. Specific PCR products were identified with ethidium bromide.

### Example 1

### Demonstration that Cell Clones Lose Protein Expression Over Time as Shown by Northern Blot Analysis

CHO cells, as described above, were transfected with a gene encoding an anti-RAGE (Receptor for Advanced Glycation Endproducts; see US20070286858) antibody and a DHFR marker gene and the cell clones were monitored over time to assess stability of gene expression. FIG. 2 is a northern blot analysis of clones that have lost protein expression. Using a DHFR specific probe, there is a loss of the HC-DHFR transcript and the emergence of a smaller DHFR only transcript. RNA from clones that lost expression of the protein of interest, but still survived methotrexate (MTX) selection was cloned and sequenced by 5′ rapid amplification of cDNA ends using the GeneRacer Kit according to the manufacturer's instructions (Invitrogen).

### Results

Sequenced cDNA isolated from these clones demonstrated a rearrangement between the lead intron of our vector and the IRES element used for translation of the second cistron. A schematic diagram of such a rearrangement is shown in FIG. 3. This allows for cell survival under selection conditions without producing the protein of interest. The effects of this rearrangement are acute and lead to rapid loss of cellular productivity (FIG. 4).

### Example 2

### Development of a High-Throughput Assay for Assessment of a Gene Rearrangement

In order to identify clones that have this specific rearrangement, a reverse transcription polymerase chain reaction (RT-PCR) assay was developed for high throughput assessment early in cell line development. This assay uses an oligo that anneals to the RNA 5′ of the lead intron and another oligo that anneals within the DHFR coding sequence (Table I). Using the QuickAccess RT-PCR kit (Promega) according to the manufacturers instructions, RNA is converted into cDNA and then amplified (Table II). A full-length, unaltered gene product will vary from product to product, but is expected to be ˜2.7 kilobases for antibody heavy chain gene products (FIG. 5). A rearranged gene product will range in size from about 300 to about 800 base pairs, or about 500 to about 700 base pairs. More specifically, a rearranged gene product, wherein the gene encoding the protein of interest is deleted, will be about 550 base pairs. The size of the rearranged product varies from clone to clone but in all cases protein of interest expression is lost.

### Results

Using this ‘loop out detection assay’ or LODA on RNA isolated from clones early in selection, a detectable RT-PCR product of ˜500 by is seen as early as day 60 post-transfection (FIG. 6).

### Example 3

### Detection of Rearrangement of Genes Encoding Various Proteins of Interest

Using the Materials and Methods described above, a study was done to determine whether this high throughput assay could be used to detect gene rearrangements in cell clones transfected with various genes encoding three different proteins of interest. Examples of this are shown in FIG. 7, whereby such analysis was done using genes encoding an Fc-fusion molecule (Fc fused to a soluble IL21 receptor; see US20060039902), a small modular immunotherapeutic product (SMIP) (an anti-CD20 SMIP; see US20030133939) and a monoclonal antibody (Mab) specific for RAGE.

### Results

FIG. 7, lane 1 (left to right), shows a clonal population of cells transfected with a gene encoding an Fc-fusion protein and shows a full length gene at day 61 post transfection, These cells should now be stable and suitable for large-scale production of the Fc-fusion protein. However, lane 2 shows another clonal population of cells that expresses a rearranged gene encoding an Fc-fusion protein at day 61 post transfection, as demonstrated by the presence of a truncated form (500 bp) of the Fc-fusion gene. These cells are not suitable for large-scale production of the Fc-fusion protein.

Lane 4 shows a clonal population of cells transfected with a gene encoding a SMIP and shows a full length gene at day 70 post transfection, These cells should now be stable and suitable for large-scale production of the SMIP. However, lane 3 shows another clonal population of cells that expresses a rearranged gene encoding the same SMIP at day 87 post transfection, as demonstrated by the presence of a truncated form (500 bp) of the SMIP gene. These cells are not suitable for large-scale production of the SMIP.

Lane 5 shows a clonal population of cells transfected with a gene encoding a monoclonal antibody (MAb) and shows a rearranged gene encoding the MAb at day 65 post transfection. These cells are not suitable for large-scale production of the monoclonal antibody. The control MAb with the full length gene expressing the Mab is not shown.

TABLE I
Oligonulceotides Used in the Loop-Out
Detection Assay (LODA) Assay

| Oligo- | |
|---|---|
| nucleotide | Sequence |
| Int GenF | 5′ TAC TCT TGG ATC GGA AAC CCG TCG 3′ |
| | (SEQ ID NO: 1) |
| DHFR2 | 5′ CTA CTT TAC TTG CCA ATT CC 3′ |
| | (SEQ ID NO: 2) |

List of oligos currently used in the loop-out detection assay. The forward oligo hybridizes to the tripartite leader sequence (Int GenF). The reverse oligo hybridizes to the mouse DHFR sequence. Although these oligos are currently in use, any complimentary oligo sequence to either of these regions, or to other regions 5′ and 3′ to the gene encoding the protein of interest, can be used.

TABLE II
Current LODA RT-PCR Conditions

| Component | Quantity |
|---|---|
| 2X QuickAccess Buffer | 25 μl |
| 50 mM Int GenF | 1 μl |
| 50 mM DHFR2 | 1 μl |
| RNA (1 μg/μl) | 1 μl |
| Reverse Transcriptase | 1 μl |
| Water | 21 μl |

List of components used in LODA RT-PCR. Used per manufacturers instructions. As oligos change to better optimize the assay, these conditions are subject to change.

### SUMMARY

One object of the present invention was to identify a clonal population of cells expressing a protein of interest, which was suitable for large-scale production of the protein of interest. Studies were done to assess whether a high-throughput screen could be developed using a method such as, but not limited to, a polymerase chain reaction (PCR) to measure the presence or absence of a rearranged gene expressing the protein of interest. If a rearrangement was observed in the gene of interest, this would indicate that the cells were not suitable for use in large-scale production of the protein of interest. The data demonstrated that such a gene rearrangement could be observed in cells expressing various proteins of interest, including an Fc-fusion protein, a SMIP and a monoclonal antibody. Moreover, when the cells expressed this genetic rearrangement, these cells were shown to be unstable and unsuitable for large-scale production of the protein of interest.

The advantage of this invention is that we can screen for clones having this rearranged transcript at any point, including early, in the cell line adaptation process. Every clone examined thus far that has developed this rearrangement has lost expression of our protein of interest. This invention is applicable to a clonal population of cells suitable for large-scale expression of any protein of interest, e.g., therapeutic proteins. The presence of the larger transcript, that is the transcript encoding the protein of interest, does not mean that a cell cannot lose expression of the protein of interest via another mechanism(s) nor does it mean that the DNA rearrangement cannot occur at a later time point during cell line development. This assay has been developed for use as a more sensitive screening method than northern blots to eliminate clones that may lose protein production. Nevertheless, the method of the invention may be used at anytime during the production of a protein of interest.

TABLE 3
Listing of Sequences

| Sequence Identifier | Description and Genbank Accession Number |
|---|---|
| (SEQ ID NO.) | where appropriate |
| 1 | Forward primer “Int Gen F” |
| 2 | Reverse primer “DHFR2” |
| 3 | Accession No. AJ000155 (pSVIRES-G vector) |
| 4 | IRES element from No. AJ000155 pSVIRES-G |
| | vector (base numbers 767 to 1310 of AJ000155) |
| 5 | Accession No. V01149 Poliovirus 1 Mahoney |
| 6 | IRES element from No.: V01149 (base numbers |
| | 1-627 of V01149) |
| 7 | Accession Number NC_001461 Bovine viral |
| | diarrhea virus |
| 8 | IRES element from No. NC_001461 (base |
| | numbers 1-900 of NC_001461) |

1. A method of identifying a clonal population of cells suitable for large-scale production of a protein of interest, the method comprising:
a) transfecting a population of cells with a nucleic acid construct comprising a gene encoding a protein of interest;
b) isolating a clonal population of cells expressing the gene encoding the protein of interest;
c) determining the presence or absence of a rearrangement of the gene encoding the protein of interest in the clonal population;
d) selecting a clonal population of cells from step c) that lack the rearrangement of the gene encoding the protein of interest; and
e) culturing the clonal population of cells from step d) for large-scale production of the protein of interest.

2. A method of identifying a clonal population of cells suitable for large-scale production of a protein of interest, the method comprising:
a) transfecting a population of cells with a nucleic acid construct comprising in sequential order a coding region for a tripartite leader sequence (TPL), an intron, a gene encoding a protein of interest, an IRES and a coding region for a selectable marker;
b) isolating a clonal population of cells expressing the gene encoding the protein of interest and the selectable marker;
c) determining the presence or absence of a rearrangement of the gene encoding the protein of interest in the clonal population;
d) selecting a clonal population of cells from step c) that lack the rearrangement of the gene encoding the protein of interest; and
e) culturing the clonal population of cells from step d) for large-scale production of the protein of interest.

3. The method of claim 1, further comprising isolating and purifying the protein of interest from the clonal population of cells.

4. The method of claim 1, wherein the large-scale production of step e) comprises culturing the cells in a volume of greater than two liters of cell culture medium.

5. The method of claim 1, wherein the nucleic acid construct comprises an intron sequence located 5′ to the gene encoding the protein of interest.

6. The method of claim 5, wherein the nucleic acid construct comprises a coding region for a tripartite leader (TPL) sequence located 5′ to the intron sequence.

7. The method of claim 1, wherein the nucleic acid construct further comprises, in sequential order, an Internal Ribosome Entry Site (IRES) operably linked to a coding region for a selectable marker, wherein the IRES and selectable marker coding region are located 3′ to the gene encoding the protein of interest.

8. The method of claim 1, wherein the gene encodes a heavy chain of an immunoglobulin molecule.

9. The method of claim 1, wherein the gene encodes a light chain of an immunoglobulin molecule.

10. The method of claim 7, wherein the selectable marker is selected from the group consisting of dihydrofolate reductase (DHFR), neomycin transferase, histidinol, hygromycin, glutamine synthetase, zeocin and phleomycin.

11. The method of claim 7 wherein the IRES is selected from the group consisting of SEQ ID NOs: 4, 6 and 8.

12. The method of claim 1, wherein the rearrangement comprises a deletion of all or part of the gene.

13. The method of claim 12, wherein the deletion is detected in a nucleic acid selected from the group consisting of DNA, pre-mRNA and mRNA.

14. The method of claim 1, wherein the gene encodes an antibody, a fusion protein, or a small modular immunopharmaceutical (SMIP).

15. The method of claim 14, wherein the antibody is a therapeutic antibody.

16. The method of claim 1, wherein the determining step comprises helicase dependent amplification or any polymerase chain reaction (PCR) selected from the group consisting of RT-PCR, inverse PCR, quantitative PCR, real-time PCR, and in situ PCR.

17. An assay for identifying a clonal population of cells suitable for large-scale production of a protein of interest, comprising:
a) culturing cells comprising a nucleic acid construct comprising a gene encoding a protein of interest to produce a clonal population of cells;
b) amplifying by polymerase chain reaction (PCR) a portion of the gene, wherein the amplification is carried out using a first primer and a second primer, wherein the first primer hybridizes to a nucleotide sequence that is 5′ to the gene, and the second primer hybridizes to a nucleotide sequence that is 3′ to the gene; and
c) determining the presence or absence of a deletion of all or part of the gene in the amplified portion of the gene;
wherein the absence of the deletion identifies the clonal population of cells as suitable for large-scale production of the protein of interest.

18. The assay of claim 17, wherein the nucleic acid construct further comprises a coding region for a tripartite leader (TPL) sequence located 5′ to the gene.

19. The assay of claim 18, wherein the nucleic acid construct further comprises, in sequential order, an Internal Ribosome Entry Site (IRES) operably linked to a coding region for a selectable marker, wherein the IRES and the selectable marker coding region are located 3′ to the gene.

20. The assay of claim 19, wherein the amplifying step comprises hybridizing the first primer to the coding region for the TPL sequence, and hybridizing the second primer to the coding region for the selectable marker.

21. A clonal population of cells suitable for large scale production of a protein of interest produced by the method of claim 1.
